(19) United States
(12) Patent Application Publication
(10) Pub. No.: US 2015/0265656 A1
(43) Pub. Date: Sep. 24, 2015

(54) HUMAN ENDOCRINE PROGENITORS FROM ADULT PANCREATIC TISSUE

(71) Applicant: THE JOHNS HOPKINS UNIVERSITY, Baltimore, MD (US)

(72) Inventor: Michael J. Shamblott, S. Pasadena, FL (US)

(21) Appl. No.: 14/434,517

(22) PCT Filed: Oct. 14, 2013
(86) PCT No.: PCT/US2013/064790

§ 371 Date: Apr. 9, 2015

Related U.S. Application Data
(60) Provisional application No. 61/712,991, filed on Oct. 12, 2012.

(51) Int. Cl. A61K 35/39; C12N 5/071; A61K 35/55; G01N 33/569

(52) U.S. Cl. CPC A61K 35/39; G01N 33/56966; C12N 5/0613; A61K 35/55; C12N 5/0676; G01N 2333/70596; G01N 2333/4703; G01N 2405/10; C12N 2533/90; C12N 2533/40; C12N 2506/07

(57) ABSTRACT

The present invention relates to the field of progenitor cells. More specifically, the present invention provides compositions and methods for isolating endocrine progenitor cells from pancreatic tissue. In certain embodiments, the method comprises the steps of (a) providing a pancreatic tissue sample; (b) isolating cells positive for CD133+; and (c) culturing the isolated cells in defined media for at least about 4 days.

### CROSS-REFERENCE TO RELATED APPLICATIONS

This application claims the benefit of U.S. Provisional Application No. 61/712,991, filed Oct. 12, 2012; which is incorporated herein by reference in its entirety.

### FIELD OF THE INVENTION

The present invention relates to the field of progenitor cells. More specifically, the present invention relates to the isolation of human endocrine progenitor cells.

### BACKGROUND OF THE INVENTION

Patients with type I (juvenile) diabetes lack pancreatic beta cells. Beta cells produce insulin, the hormone responsible for glucose homeostasis. In the absence of beta cells, type I diabetics must mechanically test and administer insulin to stay alive. Even the most diligent patients suffer significant lifetime morbidity. A great need exists for alternative methods and compositions for treating type I diabetics.

### SUMMARY OF THE INVENTION

The present invention is based, at least in part, on the discovery that human endocrine progenitor cells can be isolated from pancreatic tissue. In particular embodiments, the cells described herein are derived from adult human tissue and could be isolated from autologous tissue (from the patient). In specific embodiments, the process for generating such cells is about four days of culture in a fully defined media that is already approved for clinical use in preserving islet function prior to allotransplant.

Endocrine progenitor cells uniquely give rise to the hormone producing cells of the pancreatic islet. During development, the transcription factor neurogenin 3 (NGN3) is necessary and sufficient to generate an endocrine pancreas. The present inventors identified an endocrine progenitor cell population in adult human pancreatic tissue that expresses NGN3 and proteins that allow isolation of viable cells for clinical use. The human endocrine progenitors described herein are capable of generating cells that can produce two types of islet hormones in vitro (insulin and glucagon). If these cells behave as predicted by development, they should be capable of producing all of the endocrine cells of the pancreas.

As described herein, the present inventors demonstrate the presence of NGN3 protein-expressing cells in histologically normal duct and acinar cells in the adult human pancreas. NGN3+ cells accumulate following short-term culture of pancreatic tissue. The NGN3+ cell population is negatively regulated by Notch signaling and proteasome degradation and positively regulated by EGF receptor and STAT3 signaling. NGN3+ cells have high aldehyde dehydrogenase (ALDH) activity and coexpress the cell surface glycoprotein CD133 that can be used to obtain viable cell populations enriched for NGN3 expression (CD133+/NGN3+ cells). A subpopulation of CD133+/NGN3+ cells are capable of clonal expansion, sphere formation and differentiation into cells that release insulin C-peptide (CPEP) when exposed to elevated levels of glucose. Identification of CD133+/NGN3+ cells will facilitate studies of islet neogenesis in the adult human pancreas and potentially contribute to therapeutic strategies to treat type 1 diabetes.

In other embodiments, the progenitor cells can be differentiating by embedding the cells in a matrix/mesh construct, for example, a matrix comprised of extra cellular matrix extract from human adult islets or whole pancreas, or a synthetic hydrogel comprised of polyethylene diacrylate. The mesh can be comprised of fibers of macro or nano scale diameter. The mesh can be comprised of materials intended to persist within the body long term or to biodegrade and be reabsorbed. In certain embodiments, the fibers can comprise electrospun polycaprolactone nanofibers or fibrous material intended for surgical procedures such as for tissue support following hernia reconstruction or mesh used to stop bleeding.

In particular embodiments, CD133+ cells can be mixed in a hydrogel and used to impregnate a nanofiber mesh. In a specific embodiment, a large number of CD133+ cells are used (e.g., >5×10⁶). In another embodiment, the CD133+ cells are also ALDH+. In yet another embodiment, the cells are SSEA-4+. In other embodiments, the nanofiber mesh can be attached to standard surgical mesh in a system the looks like a bandage, wherein the nanofiber/hydrogel/cell scaffold is in the center. In such embodiments, the transplant system can be sutured to a vascular surface (e.g., the greater omentum) so that host blood cells can cross into the scaffold.

Accordingly, in one aspect, the present invention provides methods for isolating a population of endocrine progenitor cells. In certain embodiments, the method comprises the steps of (a) providing a pancreatic tissue sample; (b) isolating cells positive for CD133+; and (c) culturing the isolated cells in defined media for at least about 4 days. In particular embodiments, the isolation step is carried out using immunomagnetic beads. In a particular embodiment, the isolation step is accomplished using fluorescence-activate cell sorting (FACS). In a specific embodiment, the method further comprises selecting for Aldefluor-positive cells prior to the culturing step. In another specific embodiment, the method further comprises selecting for SSEA-4+ cells prior to the culturing step. The present invention also provides a substantially pure population of CD133+ cells isolated by the methods described herein, wherein the cells are also NGN3+.

In another embodiment, the present invention provides a population of cells comprising at least about 90% endocrine progenitor cells, wherein the progenitor cells have the phenotype CD133+. In a specific embodiment, the progenitor cells have the phenotype NGN3+. In another embodiment, the progenitor cells are ALDH+. In yet another embodiment, the progenitor cells are SSEA-4+. In a further embodiment, the progenitor cells are capable of clonal pancosphere formation.

In another aspect, the present invention provides methods for differentiating human endocrine progenitors. In certain embodiments, the method comprises the steps of (a) suspending CD133+/NGN3+ cells in a matrix; (b) mixing the cells-matrix with in a fiber mesh; and (c) differentiating the cells into CPEP+ cells. In a specific embodiment, the cells are ALDH+ and/or SSEA-4+. In another embodiment, the matrix comprises extra cellular matrix extract from human adult islet cells or whole pancreas. In yet another embodiment, the matrix comprises a synthetic hydrogel. In a further embodiment, the synthetic hydrogel comprises polyethylene glycol diacrylate. In one embodiment, the matrix comprises Matrigel. The fiber mesh can comprise fibers of micro scale. Alternatively, or in combination, the fiber mesh comprises fibers of nano scale. In certain embodiments, the fiber mesh is biodegradable. In particular embodiments, the fiber mesh comprises electrospun polycaprolactone nanofibers.

In certain embodiments, the present invention provides a substantially pure population of human endocrine progenitor cells having the following phenotype: CD133+, NGN3+, and ALDH+. In a specific embodiment, the cells are also SSEA-4+. The population of cells can further exhibit increased PTF1A expression and increased NEUROD1 expression.

In another aspect, the present invention provides methods for treating diabetes in a subject. In certain embodiments, the methods comprise the step of transplanting into the subject a population of endocrine progenitor cells made by the methods described herein. In another embodiment, a method for treating diabetes in a subject comprises the steps of (a) culturing a population of cells made by the methods described herein under conditions that differentiate the progenitors into beta cells; and (b) transplanting the beta cells into the subject.

In another aspect, the present invention provides methods for pharmacologically expanding endogenous endocrine progenitor cells as a means to treat diabetes (i.e., in vivo). In a particular embodiment, MG132 and the like can be used to expand the pool of cells in patients and the cells can be allowed to differentiate into beta cells.

### BRIEF DESCRIPTION OF THE FIGURES

FIG. 1: Expression of NEUROGENIN 3 (NGN3) in an individual adult human pancreas. A, ductal cell expression of NGN3 shown in red, cytokeratin 19 (CK19) in green, nuclei costained with Hoechst 33342 stain (H) shown in blue. B-D, higher magnification of tissue shown in A. B, CK19 expression, C, NGN3 expression, D, Hoechst stain. E, acinar cell expression of NGN3 shown in red, amylase (AMY) in green, nuclei costained with Hoechst stain shown in blue. F-H, higher magnification of tissue shown in E. F, amylase expression, G, NGN3 expression, H, Hoechst stain. A-H, NGN3+ cells indicated by white arrowheads. Scale bar is 20 μm.

FIG. 2: Regulation of NEUROGENIN 3 (NGN3) mRNA and protein. A-C, Normalized mRNA expression level of: A, HES1, B, NGN3, C, PTF1A after 4 days with Notch inhibiting γ-secretase inhibitor N—[N-(3,5-Difluorophenacetyl-L-alanyl)]-5-phenylglycine t-butyl ester (DAPT) expressed as a percentage of carrier only control (100%). D-H, % NGN3+ nuclei expressed as % carrier only control after 4 days treatment with: D, DAPT and γ-secretase inhibitor 7-amino-4-chloro-3-methoxyisocoumarin (JLK6), E, JAG-1 peptide (JAG) or scrambled JAG-1 peptide (SJAG), F, STAT3 inhibitor PpYLKTK-mts, G, EGF receptor inhibitor AG1478 and H, proteasome inhibitor MG132. Significance determined by ANOVA with Bonferroni-Holm post hoc analysis, ***, P<0.001 in A-H. I, Immunoprecipitation and western blot analysis of human adult pancreas extract and mouse E14.5 pancreatic epithelia. Inclusion of anti-NGN3 antibody and lysate in the immunoprecipitation indicated above and below blot image, respectively. Detection antibodies indicated on left. Apparent Molecular weight indicated in KDa. IgG1 heavy chain detected by secondary antibody indicated as HC.

FIG. 3: NEUROGENIN 3 (NGN3) and CD133 expression. A-H, Confocal microscopic imaging of CD133 and NGN3 expression in 2 pancreas tissue preparations after 4 days of culture. A, E, CD133 expression shown in red, B, F, NGN3 expression shown in green, C, G, nuclei costained with 4′,6-diamidino-2-phenylindole (DAPI) shown in blue. D, H, overlay of 3 channels. 1-μm optical sections. Scale bar is 50 μm. I, scatter plot of initial % CD133 (Y-axis) and % islet purity (X-axis). R²=0.79. J, change in the percentage (black bars) and total number (white bars) of CD133+ cells over time in culture as a percentage of mean±s.e.m baseline level on D2 (100%). Significance determined by ANOVA with Bonferroni-Holm post hoc analysis, ***, P<0.001, **, P<0.01, *, P<0.05 (n=6). K-M, FACS analyses of cells from pancreas tissue cultures. K, % CD133 prior to CD133-enrichment. Percent CD133 shown in upper right corner. L, CD133 expression following CD133-enrichment of cells shown in K. Percent CD133 shown in upper right corner. M,% NGN3+ cells after CD133-enrichment. Percent NGN3 shown in upper right corner. N, FACS analysis of coexpression of CD133 and ALDH. Populations P2-5 were collected. O, Normalized NGN3 mRNA expression of populations sorted for CD133 and ALDH in N. US, unsorted cells.

FIG. 4: Expression of hormones, chromogranin A and PDX1 by CD133+ cells following differentiation culture. A-F, Pancosphere differentiation. A, Orthogonal analysis of PDX1 shown in magenta, scale bar is 10 μm. B, C, Expression of insulin C-peptide (CPEP), green, glucagon (GLU) red, PDX1, white in pancospheres treated with mycophenolic acid. Scale bar is 10 μm and 100 μm in B and C, respectively. Inset in C is a higher magnification of the structure indicated by white lines. D, coexpression of CPEP (green), CgA (red) and PDX1 (magenta) by cells within a pancosphere. Scale bar is 10 μm. E, coexpression of GLU (green) and CgA (red) by cells within a pancosphere. Scale bar is 10 μm. A-E, Nuclei stained with Hoechst 33342 stain (H) shown in blue, 1 μm optical sections. F, phase microscopic image of pancospheres. Scale bar is 50 μm. G-J, biomatrix scaffold differentiation of CD133+ cells. G, I, light microscopy image of polycaprolactone (PCL) nanofiber scaffold segments with cells embedded in G, polyethylene glycol diacrylate and I, Matrigel. Scale bar is 100 μm. H, J, insulin C-peptide (CPEP) expression following endocrine differentiation. CPEP (green) and chromogranin A (CgA, red). Nuclei stained with Hoechst 33342 stain (H) shown in blue. Scale bar in H, J is 50 μm. K, Static CPEP release by cells in PCL scaffolds. CPEP levels of cells embedded in polyethylene glycol diacrylate (green triangle, red square, light blue star) and Matrigel (purple diamond, orange circle, dark blue diamond). Incubation time shown on the X-axis in minutes. Concentration of CPEP (pmol CPEP/L) shown on the left Y-axis. Concentration of glucose (mM) shown on right Y-axis and black lines above.

FIG. 5 is a FACS analysis of CD133 and SSEA-4 expression in human exocrine pancreas tissue after four days of culture. Two-channel analysis of CD133 expression: Upper left (UL) quadrant contains cells bound by CD133-PE. FIG. 5B: cells stained with both CD133-PE and SSEA-4-FITC. Cells staining for both markers are shown in the upper right (UR) quadrant. Populations were gated to display live cells. 43.2% of cells are CD133+, 42% are double positive.

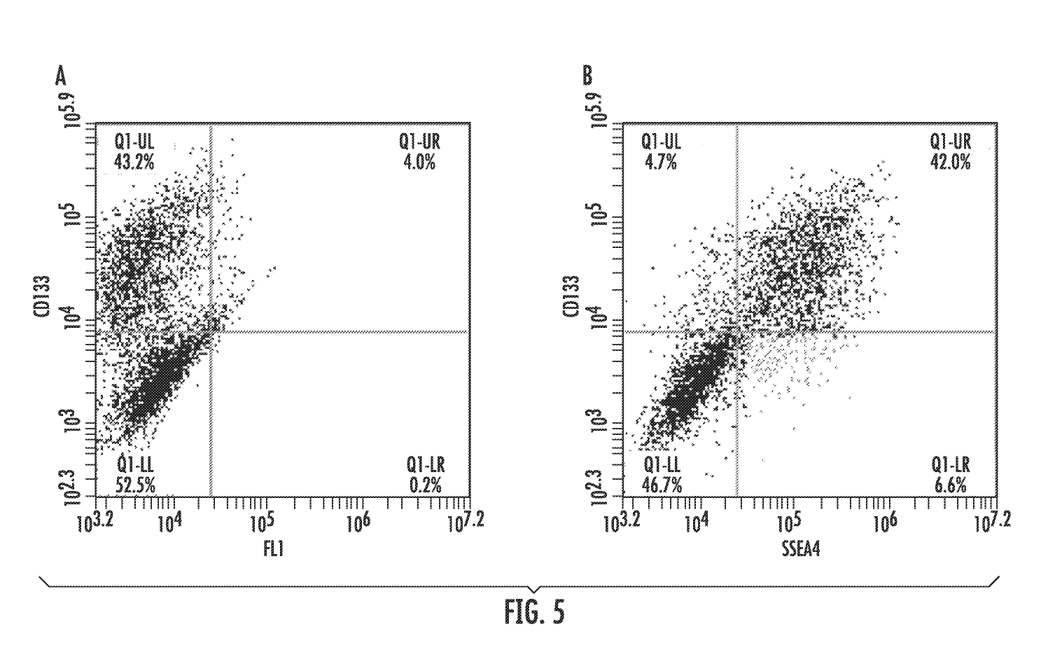

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. DEFINITIONS

The term “pancreatic cell” refers to a pancreatic islet, acinar, centroacinar, duct cell, or any other cell that is a component of the tissue in a developing or mature pancreas. Pancreatic islet cells include alpha, beta, delta, PP, and epsilon cells. Pancreatic cells or pancreatic progenitor cells can include a combination of cells found in the pancreas or of cells that develop or can develop into pancreatic tissue.

As used herein, the terms “specific binding,” “selective binding” and the like, are used interchangeably and refer to a binding reaction which is determinative of the presence of a marker, such as CD133, in a heterogeneous population of proteins, proteoglycans, and other biologics. Thus, under designated conditions, the antibodies or fragments thereof bind to a particular marker or marker fragment or variant thereof without binding in a significant amount to other proteins, proteoglycans, or other biologics present in the subject or sample.

The concept of specific or selective binding to an antibody can involve the use of an antibody that is selected for its specificity for a particular protein, proteoglycan, or variant, fragment, or protein core thereof. A variety of immunoassay formats may be used to select antibodies that selectively bind with a particular protein, proteoglycan, or variant, fragment, or protein core thereof. For example, solid-phase ELISA immunoassays are routinely used to select antibodies selectively immunoreactive with a protein, proteoglycan, or variant, fragment, or protein core thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem. 107:220 (1980).

By a “substantially pure population of cells” is meant that the cells having a selected phenotype (e.g., human endocrine progenitor cells) constitute at least about 80% of the cell population. In more specific embodiments, the cells having the selected phenotype comprise at least about 85%, at least about 86%, at least about 87%, at least about 88% at least about 89% or more of the cell population. In another specific embodiment, a substantially pure population of cells refers to cells having a selected phenotype constituting at least about 90% of the cell population. In more specific embodiments, the cells having the selected phenotype comprise at least about 91%, at least about 92%, at least about 93%, at least about 94%, at least about 95%, at least about 96%, at least about 97%, at least about 98%, at least about 99% or 100% of the cell population.

When values are expressed as approximations, by use of the antecedent “about,” the particular value is disclosed as well. The endpoints of each of the ranges are significant both in relation to the other endpoint, and independently of the other endpoint. Furthermore, where specific values are explicitly disclosed herein, that value, as well as about that value, are disclosed even if not explicitly stated. For example, if the value 10 is explicitly disclosed, then about 10 is also disclosed. When a value is explicitly disclosed, less than or equal to the value, greater than or equal to the value and possible ranges between values are also disclosed. For example, if the value 10 is disclosed then less than or equal to 10, as well as greater than or equal to 10 is also disclosed. It is also understood that, throughout the application, data are provided in a number of different formats, and these data represent endpoints and starting points, and ranges for any combination of the data points. For example, if a particular data point 10 and a particular data point 15 are disclosed, it is understood that greater than, greater than or equal to, less than, less than or equal to, and equal to 10 and 15 are considered disclosed as well as any the range between 10 and 15.

“Optional” or “optionally,” as used throughout, means that the subsequently described event or circumstance can, but may not occur, and that the description includes instances where said event or circumstance occurs and instances where it does not.

As used herein, a detectable moiety is any means for detecting an interaction between a marker and its binding moiety, thereby identifying the presence of the marker. The detectable moiety may be detected using various means of detection. The detection of the detectable moiety can be direct provided that the detectable moiety is itself detectable, such as, for example, in the case of fluorophores. Alternatively, the detection of the detectable moiety can be indirect. In the latter case, a second or third moiety reacts or binds with the detectable moiety. For example, an antibody that binds the marker can serve as an indirect detectable moiety to which a second antibody having a direct detectable moiety specifically binds.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the term also includes mammals diagnosed with diabetes.

As used herein, the term “effective,” means adequate to accomplish a desired, expected, or intended result. More particularly, a “therapeutically effective amount” as provided herein refers to an amount of a cell population of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of a cell population, necessary to provide the desired therapeutic effect, e.g., an amount that is effective to prevent, alleviate, or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### II. CELL POPULATIONS, COMPOSITIONS, AND KITS

Provided herein are populations of progenitor cells. Such progenitor cells can give rise to endocrine cells. An example population comprises at least about 80% progenitor cells, including, for example, at least about 80%, at least about 85%, at least about 90%, at least about 95%, at least about 96%, at least about 97%, at least about 98%, at least about 99% or 100% progenitor cells.

The cell populations can be relatively devoid (e.g., containing less than about 20, 15, 10, 9, 8, 7, 6, 5, 4, 3, 2, or 1%) of other cells types such as exocrine cells. Optionally, example cell populations are substantially pure populations of endocrine progenitor cells.

In specific embodiments, the endocrine progenitor cells are positive for or express high amounts relative to a control of a marker. By positive for a particular marker, for example, CD133, is meant that CD133-specific antibodies or other specific binding moieties selectively bind to the marker, such that anti-CD133 antibodies or other binding moieties can be used in cell isolation and enriching procedures.

In an example population, the endocrine progenitor cells are positive for the CD133 marker. Further provided is a population of endocrine progenitor cells, wherein at least about 80%, at least about 85%, at least about 90%, or at least about 95% of the progenitor cells are positive for CD133.

The endocrine progenitor cells of a population can be positive or express high amounts relative to a suitable control of CD133. Thus, the endocrine progenitor cells of a population can be CD133 positive (or CD133^{high}).

A selected population of the endocrine progenitor cells can be optionally cultured under conditions that cause differentiation thereof. The described populations of endocrine progenitor cells can be optionally expanded in culture to increase the total number of cells.

Furthermore, the populations of endocrine progenitor cells can be immortalized. Immortalized cells include cell lines that divide repeatedly in culture Immortalized cells are optionally developed by genetic modification of a parent cell. Moreover, the populations of endocrine progenitor cells can be genetically modified to express a protein of interest. For example, the cell can be modified to express an exogenous targeting moiety, an exogenous marker (for example, for imaging purposes), or the like. The endocrine progenitor cells of the populations can be modified to overexpress an endogenous targeting moiety, marker or the like.

In certain embodiments, the cell populations are cryopreserved. Various methods for cryopreservation of viable cells are known and can be used. See, e.g., Mazur, 1977, Cyrobiology 14:251-272; Livesey and Linner, 1987, Nature 327:255; Linner, et al., 1986, J. Histochem. Cytochem. 34(9):1123-1135; U.S. Pat. No. 4,199,022 to Senkan et al.; U.S. Pat. No. 3,753,357 to Schwartz; U.S. Pat. No. 4,559,298 to Fahy, which are incorporated by reference at least for the methods and compositions described therein).

Also provided herein are kits that include reagents that can be used in practicing the methods disclosed herein and kits comprising the cell populations taught herein. The kits can include any reagent or combination of reagents that would be understood to be required or beneficial in the practice of the disclosed methods. For example, the kits can include cell populations, as well as the buffers or compositions required to use them. Other examples of kits, include reagents for cell sorting and or detection, optionally with buffers, antibodies or compositions required to use them. The kits can also include endocrine progenitor cells and instructions to use the same in the methods described herein.

Also provided herein are populations of endocrine progenitor cells made or isolated by the methods taught herein.

### III. METHODS FOR IDENTIFYING AND ISOLATING A POPULATION OF ENDOCRINE PROGENITOR CELLS

Methods for identifying the markers that characterize endocrine progenitor cells can be based on any number of methods known in the art. Among the various methods for detecting cells expressing a specific marker, some methods are typically used if the cells are to remain viable following detection, such as for further in vitro study or for transplantation or implantation into a patient, and other methods render the identified cells less amenable to further uses in their living state, for example, in studying pathology specimens or for studies at the termination of cell based or in vivo studies. The methods herein are not so limiting and applications maintaining the viability of living cells as well as those preserving cells are fully embodied herein.

Methods of identifying the markers that characterize the progenitor cells of the present invention can be based on, by way of non-limiting examples, localizing or quantitating marker epitopes on the surface of the cells, or localizing or quantitating marker epitopes within the cytoplasm or subcellular compartments therein. Exemplary methods for the aforementioned localizing or detecting are provided below but are not intended to be limiting in any way.

Detection methods are in one embodiment based upon the detection of the binding of a binding partner to a cell expressing a marker described herein (e.g., CD133). Binding partners can be detectably labeled, or can be unlabeled but further detectable by another binding partner that is detectably labeled and binds thereto. Such uses of binding partners such as antibodies, including labeled primary antibodies and labeled lectins are known in the art. Moreover, combination systems of unlabeled primary antibodies and labeled secondary antibodies are also well known in the art. Such dual systems can also include two antibodies, lectins, avidin-biotin systems, antibodies to labels, and include amplification systems to increase the detection signal. As will be described below, such detection systems are useful not only for identifying the expression of a gene product but also in isolating cells expressing such a gene product utilizing selective binding to a matrix such as a resin or beads. The invention is not so limiting as to the means for detecting the expression of the marker(s) described herein and is inclusive of all such means.

Antibody-based detection methods are among those typically but not always used to identify expression of a protein or an epitope thereof by cells, regardless of whether cells require viability during or after detection. The antibody can be a monoclonal or polyclonal antibody. Ready guidance from the literature can be followed to prepare such antibodies that specifically bind to a marker(s) on the cell surface, and can be used on living cells to detect markers on the cell surface, or in sectioned cells or tissue specimens to detect markers on the surface.

In one embodiment, the term “antibody” includes complete antibodies (e.g., bivalent IgG, pentavalent IgM) or fragments of antibodies in other embodiments, which contain an antigen binding site. Such fragment include in one embodiment Fab, F(ab′)₂, Fv and single chain Fv (scFv) fragments. In one embodiment, such fragments may or may not include antibody constant domains. In another embodiment, F(ab)'s lack constant domains which are required for complement fixation. scFvs are composed of an antibody variable light chain (V_{L}) linked to a variable heavy chain (V_{H}) by a flexible linker. scFvs are able to bind antigen and can be rapidly produced in bacteria. The invention contemplates antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies useful in the present invention can be monoclonal antibodies (Mab) in one embodiment, or polyclonal antibodies in another embodiment. Antibodies which are useful for the methods described herein can be from any source, and in addition may be chimeric. In one embodiment, sources of antibodies can be from a chicken, mouse, rat, sheep, goat, horse, or a human in other embodiments. Secondary antibodies are typically antibodies that bind to another antibody, and are typically prepared in a species different from the originating species of the primary antibody, such that, for example, the secondary antibody may be a rat anti-mouse antibody, or a goat anti-rat antibody, or vice versa, e.g., mouse anti-rat antibody. In some cases a secondary antibody may be directed against a moiety conjugated to the primary antibody, such as a fluorescent moiety. In other embodiments, other binding partners such as avidin and biotin may be employed. In certain embodiments, a detectable primary antibody is used in the detection. In other embodiments, and in particular where amplification of the detectable signal that indicates the presence of the marker is needed, secondary antibodies or even further amplification techniques can be used to increase the detectability of the extent of binding of the primary antibody can be employed. Such amplification systems are well known in the art.

The detection agent described herein can be a lectin or combination of lectins selected or designed to specifically bind to a marker, e.g., CD133. These lectins can be in solution, detectably labeled or detected or retrieved by a secondary detection antibody or preferably, be attached to a solid substrate such as a magnetic bead or other surfaces that can be used to retrieve cells.

Detection of antibody binding to a cell typically requires a detectable label, either directly bound to the marker-binding antibody (primary antibody) itself, or the detectable label can be present on a secondary antibody that binds to the primary antibody. Various detectable labels are embodied herein, and the selections are not intended to be limiting. Labels such as fluorescent moieties, radioactive elements and compounds, and proteins or other entities with enzymatic activity have been used in the art and are well known, and are applicable to different methods of detection. In one embodiment, among useful fluorescent labels is phycoerythrin. In another embodiment, radioactive labels include ¹²⁵I.

As noted above, the term “detectable label” or “detectably labeled” refers in one embodiment to a composition or moiety that is detectable by spectroscopic, photochemical, biochemical, immunochemical, electromagnetic, radiochemical, or chemical means such as fluorescence, chemifluoresence, or chemiluminescence, or any other appropriate means. In another embodiment, detectable labels are fluorescent dye molecules, or fluorophores, such fluorescein, phycoerythrin, CY3, CY5, allophycocyanine, Texas Red, peridenin chlorophyll, cyanine, FAM, JOE, TAMRA, TET, and VIC, by way of non-limiting examples.

For example, Miltenyi Biotec (Auburn, Calif.) sells antibody-based reagents for identification and isolation of CD133 expressing cells; antibodies include clone AC133 (mouse IgG1), 293C3 (mouse IgG2b), and AC141 (mouse IgG1). These antibodies recognize two different epitopes CD133/1 (clone AC133) and CD133/2 (clone 293C3 and clone AC141), respectively, on the CD133 molecule. Antibody-based reagents for the identification and isolation of the other markers described herein are also commercially available.

Thus, in one embodiment, a labeled primary antibody that binds to a marker, or a combination of an unlabeled primary antibody that binds to a marker and a labeled secondary antibody that binds to the unlabeled primary antibody, can be used to identify marker expressing cells. In another embodiment, a phycoerythrin-conjugated antibody to a marker is used. Using a fluorescent label such as phycoerythrin (PE), marker-expressing cells can be identified using fluorescence microscopy. In other embodiments, a biotinylated primary antibody and a detectable reagent that binds to biotin, such as a fluorescent- or enzyme-conjugated streptavidin or other avidin derivative, can be used for fluorescence localization, immunohistochemical localization or detection by light microscopy. As will be seen below, an advantage of using phycoerythrin is that it is both detectable (fluorescent), and an antibody can be raised thereto, the anti-phycoerythrin antibody useful as an affinity reagent to isolate cells to which phycoerythrin is bound, via for example using the aforementioned phycoerythrin-conjugated anti-marker antibody. The anti-phycoerythrin antibody can be of the same species or of a different species as the primary anti-marker antibody.

In yet another embodiment, localization of marker-expressing cells in a cellular or tissue sample can be performed using immunohistochemical techniques whereby, for example, whole cells or thin sections of tissue are stained with reagents that identify marker epitopes, such as antibodies as described above either directly labeled or by using a labeled secondary antibody that produces a visible product, for example, through an enzymatic reaction, at the sites of the marker. Such immunohistochemical localization methods are well known in the art and can be readily applied to the markers described herein.

Sources of pancreatic cells for the methods described herein include pancreatic islet preparation, i.e., cells isolated from the islets of human or other species pancreata, or cells prepared from human pancreatic tissue. Tissues from adults as well as those from fetal sources are embraced herein. Pancreatic islet cell preparations, which comprise islets and exocrine tissue, can be obtained from any of a number of academic and/or clinical islet purification services. For patients undergoing pancreatectomy for the purpose, for example, of treatment of pancreatitis, the patient's own resected pancreas tissue can provide the source of cells from which pancreatic endocrine progenitor cells can be isolated by the methods embodied herein then administered to the same patient, or to another patient for the treatment of, for example, diabetes mellitus. And likewise, a pancreatectomy patient can be administered autologous endocrine progenitor cells from a single unrelated individual or a pool of individuals.

In one embodiment, endocrine progenitor cells herein are cells from the adult human pancreas that express CD133^{hi}.

The aforementioned exemplary methods for identifying cells expressing the markers described herein, and in particular methods that do not impact the viability of the cells, readily lend themselves to methods for isolating from a mixed cellular population cells that express the markers. Thus, in another embodiment, marker-expressing cells are isolated from or enriched within a mixed cellular population, utilizing various methods of detecting the expression of the markers on the cell surface. By way of non-limiting examples, fluorescence activated cell sorting technology can be used. The various reagents mentioned above useful for identifying cells expressing marker(s) in pancreatic tissue are also useful as reagents for separating such cells from a mixed cellular population, such as by binding to a solid matrix or using magnetic bead technology. Anti-marker antibodies are but one example of the use of a marker binding partner for isolating or separating marker-expressing cells.

Thus, in one embodiment, fluorescence activated cell sorting (FACS) techniques can be used to isolate cells expressing the marker(s), using either a primary anti-marker antibody conjugated to a fluorescent moiety, or an unlabeled or nonfluorescently-labeled primary anti-marker antibody and a secondary antibody conjugated to a fluorescent moiety or a fluorescent reagent that binds to the primary antibody or by using a lectin that recognizes a marker glycan (e.g., CD133). Other binding pairs such as biotin and avidin can be used to achieve the same desired cell labeling. FACS methodology is well known in the art.

In another embodiment, cells expressing CD133 can be directly isolated from a mixed population using a matrix or surface to which an antibody to a marker is conjugated, such that marker-expressing cells bind to the matrix or surface, non-adherent cells can be washed away, and the marker-expressing cells eluted from the matrix or surface. In one embodiment, a matrix such as agarose or Sepharose in the form or beads can be conjugated with antibodies to a marker. Marker-expressing cells in a mixed population are exposed to the matrix, by admixing therewith or passage through a column thereof, to which marker-expressing cells adhere, then the matrix can be washed and the cells eluted therefrom using a high salt or low pH elution buffer, or other methods that interfere with antibody-epitope interaction or methods that act to cleave the connection between the bead and desired cell type. Such methods and reagents therefor are well known in the art. In another embodiment, magnetic beads to which anti-marker antibodies are conjugated are used to bind marker-expressing cells, after which the beads are separated based on their magnetic properties, washed and the marker-expressing cells eluted therefrom. Such magnetic beads are available from Miltenyi Biotec, and methods of use described in the manufacturer's instructions. In yet another embodiment, agarose or Sepharose beads to which lectins are attached are used to bind marker-expressing cells (e.g., CD133-expressing cells). In such embodiments, CD133 expressing cells in a mixed population are exposed to the matrix, by admixing therewith or passage through a column thereof, to which CD133 expressing cells adhere, then the matrix can be washed and the cells eluted therefrom using an unconjugated glycan to that interferes with the CD133-lectin interaction or methods that act to cleave the connection between the bead and desired cell type. Such methods and reagents therefor are well known in the art.

In other embodiments, matrix or magnetic bead separation can be achieved using a secondary antibody conjugated to the matrix or beads, the secondary antibody directed against a primary antibody that binds to a marker. For example, in one embodiment, after use of a primary antibody that binds to a marker that is labeled with phycoerythrin, magnetic beads or a matrix conjugated with an antibody that binds to phycoerythrin can used to bind marker-expressing cells, after which the beads can be washed and the marker-expressing cells released. For example, Miltenyi Biotec sells magnetic beads conjugated to an anti-phycoerythrin antibody (Anti-PE microbeads). Alternately, a secondary antibody against the primary antibody molecule can be used. There methods are merely illustrative of affinity procedures and variations thereof are well known in the art and are fully embraced herein.

In embodiments of the methods for isolation of or enrichment for endocrine progenitor cells from a cellular population, the cellular population can be obtained from a pancreatic islet preparation, or from human pancreatic tissue. As noted above, pancreatic islet cell preparations can be obtained from any of a number of academic and/or clinical islet purification services. Adult as well as fetal tissues are embraced herein.

In any of the embodiments described herein, the isolated or enriched endocrine progenitor cells expressing cells can be cultured or expanded in vitro prior to any of the various uses described herein, among others, in order to, by way of non-limiting example, expand or increase the population of cells.

### IV. METHODS OF TREATMENT

Provided herein are methods of treating diabetes in a subject. The methods can include the step of transplanting into the subject a population of endocrine progenitor cells made by the methods taught herein or using a population of endocrine progenitor cells taught herein. The methods can also include culturing a selected population of endocrine progenitor cells under conditions that cause differentiation thereof. The resulting differentiated cells, or a subset thereof, can then be transplanted into the subject in need of treatment (e.g., diabetes). In alternative embodiments, the present invention provides methods for pharmacologically expanding endogenous endocrine progenitor cells as a means to treat diabetes (i.e., in vivo). In a particular embodiment, MG132 and the like can be used to expand the pool of cells in patients and the cells can be allowed to differentiate into beta cells.

The number of progenitor cells or differentiated cells transplanted can range from about 10² to about 10⁸ at each transplantation (e.g., injection site), depending on the size and species of the recipient. Single transplantation (e.g., injection) doses can span ranges of about 10³ to about 10⁵, about 10⁴ to about 10⁷, and about 10⁵ to about 10⁸ cells, or any amount in total for a transplant recipient patient.

Delivery of the cells to the subject can include either a single step or a multiple step injection. The cellular transplants are optionally injected as dissociated cells but can also be provided by local placement of non-dissociated cells. In either case, the cellular transplants optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated cellular transplant can be a streaming injection made across the entry path, the exit path, or both the entry and exit paths of the injection device (e.g., a cannula, a needle, or a tube). Automation can be used to provide a uniform entry and exit speed and an injection speed and volume. Optionally a multifocal delivery strategy can be used. Such a multifocal delivery strategy is designed to achieve widespread and dense donor cell engraftment throughout the recipient. Injection sites can be chosen to permit contiguous infiltration of migrating donor cells into particular areas.

In yet another embodiment of the invention, methods are provided for treating a patient having diabetes mellitus using the endocrine progenitor cells isolated from a cellular population in accordance with, and by way of non-limiting examples, the embodiments described above, then administering the progenitor cells to the patient. In one embodiment the cells are cultured or expanded in vitro prior to use. In other embodiments, the cells are differentiated in vitro prior to use.

For example, the endocrine progenitor cells isolated or enriched in accordance with the embodiments herein can be directly injected into the hepatic duct or the associated vasculature of a patient. In another embodiment the cells can be cultured and expanded in vitro prior to injection. Similarly, cells can be delivered into the pancreas by direct implantation or by injection into the vasculature. Cells engraft into the liver or pancreatic parenchyma, taking on the functions normally associated with pancreatic cells, respectively. Moreover, before implantation or transplantation the cell obtained as described herein can be genetically manipulated to reduce or remove cell-surface molecules responsible for transplantation rejection in order to generate universal donor cells. For example, the mouse Class I histocompatibility (MHC) genes can be disabled by targeted deletion or disruption of the beta-microglobulin gene (see, e.g., Zijlstra, Nature 342:435-438, 1989). This allows indefinite survival of murine pancreatic islet allografts (see, e.g., Markmann, Transplantation 54:1085-1089, 1992). Deletion of the Class II MHC genes (see, e.g., Cosgrove, Cell 66:1051-1066, 1991) further improves the outcome of transplantation. The molecules TAP1 and Ii direct the intercellular trafficking of MHC class I and class II molecules, respectively (see, e.g., Toume, Proc. Natl. Acad. Sci. USA 93:1464-1469, 1996); removal of these two transporter molecules, or other MHC intracellular trafficking systems may also provide a means to reduce or eliminate transplantation rejection. Such techniques can be applied to human cells and the corresponding HLA antigens. In another embodiment, the cellular population is obtained from a pancreas HLA matched to the subject.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Pancreatic Tissue Culture.

Human cadaveric pancreas tissue that had been enzymatically digested and subjected to density centrifugation (57) was received 2-3 days post mortem. In all experiments, stated replication refers to the number of cultures from individual organ donors unless otherwise defined. Islet purity varied between preparations from <10% to >90%. Pancreatic tissue was resuspended in 10 ml Miami Media 1A (Mediatech) supplemented with freshly prepared 0.01 g/L glutathione and cultured in low adhesion dishes at 37° C. for 4 days. Media was replaced every 2-3 days. Inhibition of γ-secretase was carried out by daily addition of N—[N-(3,5-Difluorophenacetyl-L-alanyl)]-S-phenylglycine t-butyl ester (DAPT, Tocris Biosciences) or 7-Amino-4-chloro-3-methoxyisocoumarin (JLK6, EMD Biosceinces). JAG-1 (ANASPEC), a 17-residue polypeptide corresponding to amino acids 188-204 of the Jagged-1 Notch ligand (58) and a negative control polypeptide comprised of scrambled JAG-1 amino acids were used at 47 μM. A second Notch ligand, Delta-like ligand 4 (D114)(R&D Systems) was used at 500 ng/ml. Inhibition of proteasome degradation was carried out with N-[(Phenylmethoxy)carbonyl]-L-leucyl-N-[(1S)-1-formyl-3-methylbutyl]-L-leucinamide (MG132, Tocris Bioscience) and (2R,3S,4R)-3-Hydroxy-2-[(1S)-1-hydroxy-2-methylpropyl]-4-methyl-5-oxo-2-pyrrolidinecarboxy-N-acetyl-L cysteine thioester (clasto-lactacystin beta-lactone, Tocris Bioscience). Inhibition of EGF receptor kinase was carried out with N-(3-Chlorophenyl)-6,7-dimethoxy-4-quinazolinanine hydrochloride (AG1478, Tocris Bioscience) and with N-(3-Chlorophenyl)-6,7-dimethoxy-4-quinazolinanine hydrochloride (Erlotinib, Cayman Chemical). STAT3 activation was blocked with a cell-permeable analog of the STAT3-SH2 domain-binding phosphopeptide (PpYLKTK-mts, EMD Biosciences).

Magnetic Bead Isolation, FACS and Aldehyde Dehydrogenase Assay.

Tissue rinsed in calcium/magnesium-free PBS was dissociated by incubation in 0.05% trypsin/EDTA for 5 min. at 37° C. Following trypsin neutralization and DNA removal by incubation with 120 units DNase I per ml at room temperature for 2 min., single cells were collected by centrifugation at 200×g for 5 min. Cells were resuspended in degassed 0.5% BSA, 2 mM EDTA prepared in calcium/magnesium-free PBS and counted by using a Nucleocounter (New Brunswick Scientific). For FACS analyses, primary antibodies were CD133/1 and CD133/2. Isotype negative control antibodies were IgG1-PE and IgG2b-PE (BD Bioscience). CD133 expressing cells were purified by using CD133/1 conjugated to magnetic beads (Miltenyi Biotec). Two rounds of magnetic bead enrichment were performed. Purification efficiency and characterization was determined by staining with CD133/2 conjugated to phycoerythrin (CD133/2-PE). Total cell number was determined in triplicate. The number of CD133 cells was calculated as total cell number x % CD133+. For aldehyde dehydrogenase (ALDH)/CD133 FACS analyses, cells were resuspended in Aldefluor (Aldagen) buffer at 1×10⁶ cells per ml and incubated 40 min at 37° C. then CD133/1-PE was added and cells were incubated 30 min at 4° C. Cells were resuspended in ice cold Aldefluor buffer and kept on ice until FACS analyses. Baseline gate parameters were established for CD133/1-PE and ALDH with isotype negative and DEAB reacted controls, respectively. For FACS analysis of NGN3, CD133+ cells were fixed in 2% PFA for 10 min at room temperature, then incubated for 30 min. at 4° C. in anti-NGN3 diluted in PBS, 2% fetal calf serum, 0.3% Triton X-100. Cells were washed in PBS, stained with secondary antibody, washed and resuspended for FACS analysis.

Culture of CD133+ Cells.

Cells were enriched using anti-CD133 immunomagnetic beads to >90% purity from cultured pancreatic tissue preparations on D7. For adherent culture, CD133+ cells were plated at 2×10⁴ cells/cm² on 48-well plates coated in 10 μg/ml type I collagen in the presence of 40 ng/ml human recombinant leukemia inhibitory factor (hrLIF), 50 ng/ml human recombinant epidermal growth factor (hrEGF) and 50 μg/ml G418. Adherent cells were quantified after 12 hrs and isolated single cells were marked for subsequent photographic analysis of cell division. Media was replaced every 2-3 days and cells were fixed after 8 days.

To form pancospheres, CD133+ cells were resuspended at 2×10⁵ cells/ml in HuES media [Knockout DMEM (Invitrogen), 10% Knockout Serum Replacement (Invitrogen), 10% Albumax, 10 ng/ml fibroblast growth factor 2 (FGF2), 1×Na pyruvate, and 1× non-essential amino acids] conditioned by overnight incubation on human SDEC cells (59, 60) and 1-2% Matrigel. Media was supplemented every 3-4 days for up to 21 days. Media was then changed to Maturation media [RPMI 1640, 0.5% BSA, 10 mM nicotinamide, 50 ng/ml human insulin like growth factor II (IGF II, R&D Systems)] for 7 days with addition of fresh nicotinamide and IGF II every 2 days without media change. IGF II was then slowly withdrawn by subsequent 50% media changes. When noted, maturation media was supplemented with 10 μM mycophenolic acid.

Clonogenic Pancosphere Assay.

CD133+ and CD133− cells were resuspended at 10, 100, 1000 cells per ml in SDEC conditioned media+1% Matrigel and 0.1 ml plated into each well of an ultra low attachment (Corning) 96 well plate. Wells containing one cell were highlighted for subsequent analysis. After 4 days, wells containing PS were counted.

Electrospun Polycaprolactone Nanofiber Biomatrix Scaffolds.

Polycaprolactone (PCL) (MW 80,000) polymer solution was solvated in 80/20 dichloromethane/methanol was placed in a motorized syringe fitted with a 5 cm, 20G blunt tipped needle. Electrode gap width was fixed at 10 cm. Electrospun fibers were collected in 95% Ethanol then chilled in liquid nitrogen. Individual constructs were produced with a 4 mm punch biopsy, washed in 70% ethanol then washed in PBS before use in cell culture. For polyethylene glycol diacrylate (PEG) constructs, 10% PEG (MW 3400) precursor solution mixed with Irgacure D2959 photoinitiatior, electrospun fibers and 5-10⁵-1×10⁶ CD133+ cells were placed into a 5 mm diameter, 3 mm deep cylindrical mold and exposed for 5 min to 365 nm UV light. Matrigel constructs were identical except Matrigel was substituted for PEG and incubated at 37° C. to polymerize.

Static CPEP Release Assay.

Scaffold segments (<40 mg) were incubated in Krebs Ringers HEPES BSA (KRHB) buffer (119 mM NaCl, 10 mM HEPES, 4.7 mM KCl, 2.5 mM CaCl₂, 1.2 mM MgCl₂, 1.2 mM KH₂PO₄, 0.1% BSA) supplemented to 2 mM glucose for 30 min. then moved into wells of a 48-well plate containing 0.2 ml KRHB with 2 mM glucose for 1 hr, 20 mM glucose sampled at 15, 30 and 60 min, then back to KRHB with 2 mM glucose for 1 hr. Samples were analyzed using the Ultrasensitive CPEP ELISA (Mercodia). Scaffold segments were returned to maturation media for post ELISA histological analysis.

Quantitative RT-PCR.

Human adult and fetal pancreas RNA was commercially prepared. RNA from islet preparation tissue and cells were prepared using the RNeasy miniprep kit (Qiagen). Synthesis of cDNA was performed by using oligo (dT) primers in a standard reverse transcriptase reaction. Levels of HES 1, PTF1A, PDX1, NGN3 and CYCLOPHILLIN A were determined by using Taqman® gene expression assays Hs00172878_m1, Hs00603586_g1, Hs00426216_m1, Hs00360700_g1 and Hs99999904_m1, respectively. Mean levels (6 readings/sample) of genes of interest were normalized to mean levels of CYCLOPHILLIN A. MicroRNAs hsa-mir-15a, hsa-mir-15b, hsa-mir-16 and hsa-mir-195 levels were measured using sequence specific reverse transcriptase primers and TaqMan® detection reagents (Applied Biosystems). Mean levels (6 readings/sample) of miRNAs were normalized to mean levels of 18S rRNA (Applied Biosystems Ribosomal RNA Reagent). Significance of RTPCR data was determined by ANOVA with Bonferroni-Holm Post hoc testing.

MicroRNAs (miRNA) hsa-mir-15a, hsa-mir-15b, hsa-mir-16 and hsa-mir-195 levels were measured using sequence specific reverse transcriptase primers and TaqMan® detection reagents (Applied Biosystems). Mean levels (6 readings/sample) of miRNAs were normalized to mean levels of 18S rRNA (Applied Biosystems Ribosomal RNA Reagent). Significance of RTPCR data was determined by ANOVA with Bonferroni-Holm post hoc testing.

Immunochemistry.

Tissues stained for NGN3 expression were sectioned and stained as soon as possible after harvest. Freshly sectioned 5-8 micron frozen sections of islet preparation tissue were fixed for 5 min. in 4% paraformaldehyde (PFA) prepared in phosphate buffered saline (PBS), quenched for 5 min in 50 mM glycine in PBS then blocked in 5% donkey serum, 1% BSA in PBS for 30 min. at room temperature. 0.1 to 0.2% Triton-X100 was included for cytoplasmic and nuclear antigens, respectively. Primary antibodies were diluted in blocking buffer; mouse anti-CD133/1 (IgG1) and CD133/2 (IgG2b) unconjugated and conjugated to phycoerythrin (Miltenyi Biotec), mouse anti-cytokeratin 19 (CK19, Chemicon), guinea pig anti-swine insulin (Dako), rabbit anti-CPEP (LINCO), mouse anti-GLU (R&D Systems), goat anti-PDX1 (gift from Chris Wright), mouse anti-CgA (Dako); goat anti-amylase (AMY, Santa Cruz), mouse anti-NGN3 (Developmental Studies Hybridoma Bank, Iowa City, Iowa, hybridoma supernatant), rabbit anti-cleaved caspase-3 (Cell Signaling Technology), rabbit Ki67 (abcam), mouse anti-human E-cadherin (Invitrogen). BrdU and EdU detection were by kit from BD Bioscience and Invitrogen, respectively. Quantitative analysis of protein expression on frozen sections was carried out by imaging >800 nuclei in randomly selected fields for each treatment group. For analysis of CD133+ cells immediately after preparative fluorescent activated cell sorting (FACS), cells were immobilized, fixed and quenched directly on glass slides using a cytospin. For identification and quantification cells within pancospheres, spheres were fixed and stained similarly to tissue sections except all incubations and washes were overnight at 4° C. Stained pancospheres were mounted on coverslipped glass slides. For quantitative analyses, unbiased stereology (Stereo Investigator software) was used to estimate number of nuclei per pancosphere. Sectioned tissue and pancospheres were imaged by confocal microscopy through the Z-axis in a series of 1 micron optical sections using sequential acquisition mode. Immunoreactive cells were counted directly and were considered positive only if confocal orthogonal analysis demonstrated immunostaining that clearly surrounded a nucleus (cytoplasmic antigens) or if immunostaining colocalized cleanly within nuclear counterstain (nuclear antigens). Mean % CPEP, % GLU and % CgA were calculated from a total of 17 pancospheres.

Immunoprecipitation. Collagenase-digested human adult pancreas tissue (˜24 hrs post mortem) and EX.X mouse fetal pancreatic epithelia was extracted in RIPA extraction buffer (0.05M Tris-HCl, pH 7.4, 0.15M NaCl, 0.25% deoxycholic acid, 1% NP-40, 1 mM EDTA) supplemented with HALT protease and phosphatase inhibitor (Thermo Scientific). Approximately 1 mg of human pancreas extract and extract-free negative control were incubated overnight with 5 μg anti-NGN3 4° C. Bound proteins were isolated with magnetic beads covalently bound to Protein G (DYNAL) Immunoprecipitated proteins and ˜20 μg E14.5 mouse pancreatic epithelia extract were resolved on 12% HEPES/glycine/SDS gels. Western blots were blocked in 5% nonfat dry milk in PBST (PBS+0.1% Tween-20) then incubated with anti-NGN3 (1:50, 5% BSA in PBST) then re-probed with anti-human E12/47 (Santa Cruz, (1:200, 5% BSA in PBST). Bound proteins were detected with anti-mouse IgG1 heavy chain-specific secondary antibody conjugated to IRDYE-800 (Licor), anti-mouse IgG light chain-specific secondary antibody conjugated to Alexa Fluor 647 (Invitrogen) or anti-rabbit Dylite 680 (Thermo Scientific) in Licor blocking buffer with 0.1% Tween-20, 0.05% SDS) or and imaged on a Licor Odyssey imager.

### Example 1

### NGN3 is Expressed by Acinar and Duct Cells in the Adult Human Pancreas

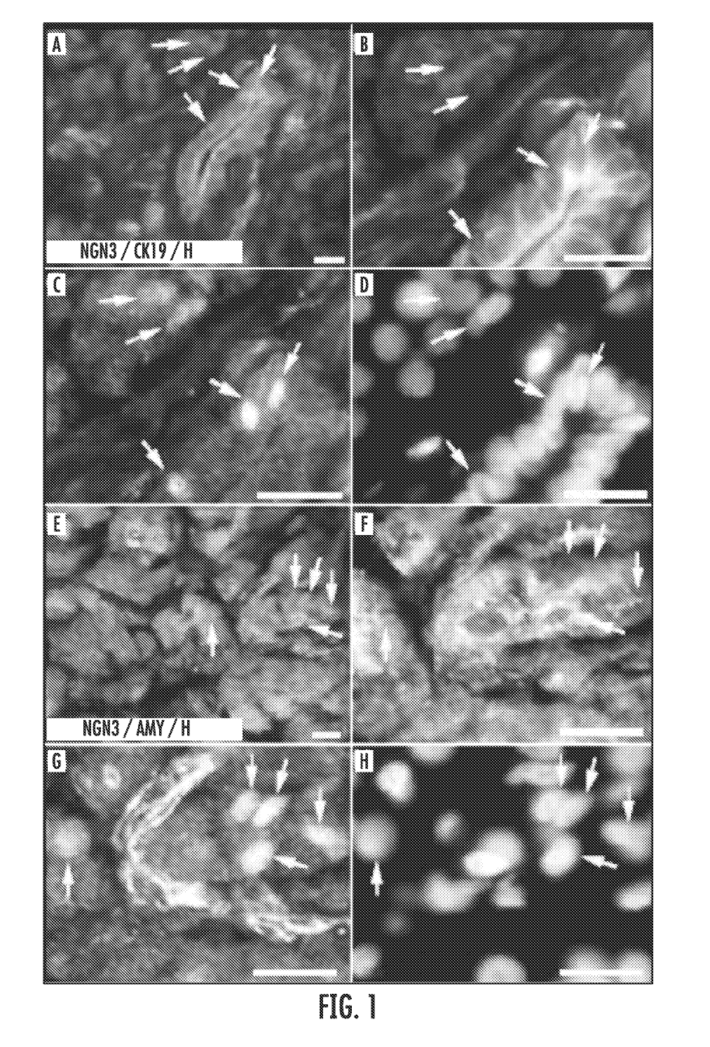

NGN3 expression was detected in grossly and histologically normal tissues from five surgically resected pancreata. In all five pancreata, NGN3 was localized in the nucleus of amylase (AMY)+ acinar cells and cytokeratin 19 (CK19)+ duct cells (FIG. 1A-H). The level of NGN3 expression varied between patients and was both heterogeneous and spatially restricted within individual tissue sections. In randomly selected fields of >100 nuclei, the mean±s.e.m % NGN3+ cells was 2.4±1.1%. Exocrine NGN3 expression was identified in an additional 15 patient biopsies.

### Example 2

### NGN3 Protein and Neurogenic Differentiation 1 (NEUROD1) mRNA Expression Increases During Culture of Pancreatic Tissue

Enzymatically-digested adult human cadaveric pancreas preparations containing a mixture of exocrine tissue and islets (pancreatic tissue) were received two to three days post mortem (D2-D3) and cultured in defined media for 4 days. This culture system was used to investigate changes in the expression of NGN3 in tissue undergoing ADM. In three independent (different organ donors) pancreatic tissue preparations, the mean±s.e.m percentage of cells expressing AMY on D7 decreased significantly to 7.3%±3.4 relative to D3 (P<0.001, n=3). The mean±s.e.m percentage of cells expressing CK19 relative to D3 increased significantly to 180.7%±19.6 (P<0.01, n=3).

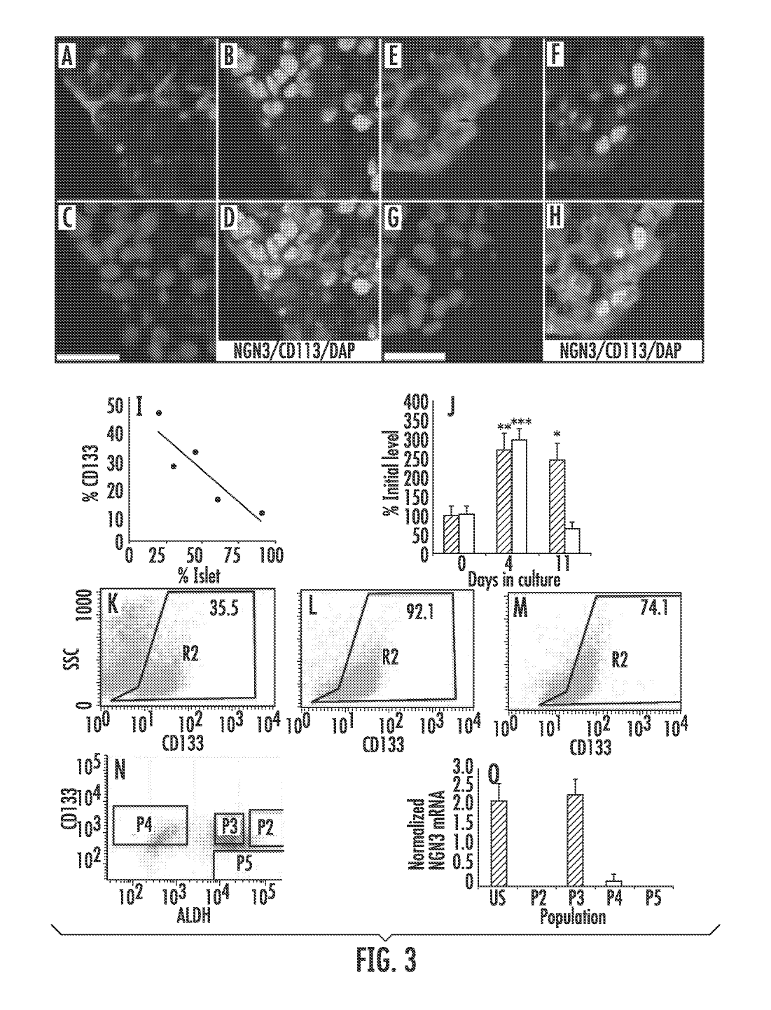

The percentage of cells expressing nuclear NGN3 protein (representative images of NGN3 expression in cultured tissue in FIG. 3B, F) increased significantly after 4 days in culture compared to initial levels (mean±s.e.m 863.1%±453.5, n=5) (Table 1). NEUROD1 is a direct transcriptional target of NGN3, and like NGN3, can drive ectopic endocrine development (24). NEUROD1 mRNA levels were significantly elevated in all 5 cultures compared to initial levels (mean±s.e.m 241.1%±31.8, n=5). However, NGN3 mRNA expression was not increased consistently following 4 days of culture.

TABLE 1
Quantification of NEUROGENIN 3 (NGN3) and NEUROD1 expression in cultured pancreas tissue
from 5 organ donors (coded A-E). Day column refers to days post mortem. Mean ± s.e.m
levels and % change compared to initial level are reported. Significance determined by ANOVA
with Bonferroni-Holm post hoc analysis. mRNA levels normalized to level of CYCLOPHILLIN A.

| | | Normalized NGN3 mRNA | | | % NGN3 + Nuclei | | | Normalized NEUROD1 mRNA | | |
|---|---|---|---|---|---|---|---|---|---|---|
| Sample | Day | Mean ± SE | % change | P value | Mean ± SE | % change | P value | Mean ± SE | % change | P value |
| A | 2 | 0.071 ± 0.002 | | | 12.2 ± 2.8 | | | 3.438 ± 0.020 | | |
| | 6 | 0.117 ± 0.003 | 164.8 | <0.01 | 28.4 ± 9.4 | 232.8 | <0.05 | 4.309 ± 0.065 | 125.3 | <0.01 |
| B | 3 | 0.021 ± 0.001 | | | 1.6 ± 1.1 | | | 0.411 ± 0.003 | | |
| | 7 | 0.021 ± 0.001 | 100.0 | >0.05 | 6.4 ± 1.3 | 400.0 | <0.05 | 1.268 ± 0.012 | 308.5 | <0.001 |
| C | 3 | 0.238 ± 0.004 | | | 7.4 ± 4.1 | | | 4.527 ± 0.123 | | |
| | 7 | 0.124 ± 0.003 | 52.1 | <0.001 | 46.2 ± 4.2 | 624.3 | <0.001 | 10.321 ± 0.132 | 228.0 | <0.001 |
| D | 2 | 0.011 ± 0.001 | | | 13.2 ± 3.5 | | | 5.725 ± 0.029 | | |
| | 6 | 0.047 ± 0.002 | 427.3 | <0.001 | 52.6 ± 10.2 | 398.5 | <0.001 | 14.915 ± 0.174 | 260.5 | <0.001 |
| E | 3 | 0.013 ± 0.001 | | | 0.5 ± 0.2 | | | 0.407 ± 0.010 | | |
| | 7 | 0.016 ± 0.001 | 123.1 | >0.05 | 13.3 ± 4.1 | 2660.0 | <0.05 | 1.152 ± 0.015 | 283.0 | <0.001 |

### Example 3

### NGN3 is Negatively Regulated by Notch Signaling

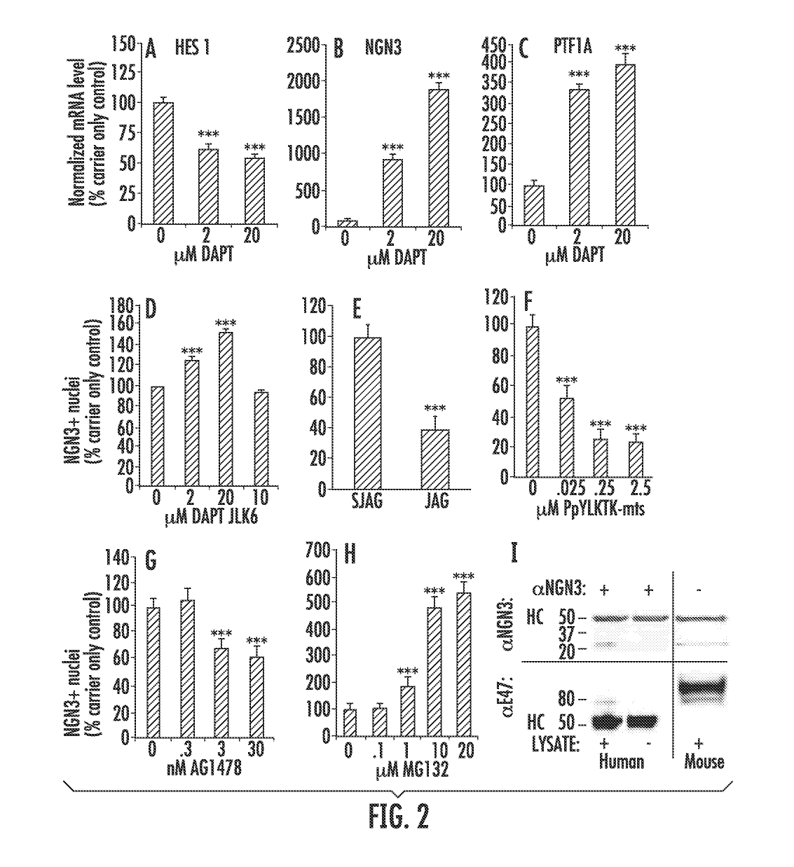

In order to investigate the involvement of Notch signaling in cultured pancreatic tissue, Notch was inhibited with the γ-secretase inhibitor DAPT (25) and the mRNA levels of the Notch downstream negative regulator of NGN3, Hairy and Enhancer of Split 1 (HES1) (1, 26, 27) were determined as was the mRNA and protein levels of NGN3. The level of HES1 mRNA decreased significantly after 4 days of culture in the presence of 2 μM and 20 μM DAPT to 63.0%±3.3 (P<0.001, n=6 readings) and 55.4%±1.5 (P<0.001, n=6 readings) of carrier control, respectively (FIG. 2A). The level of NGN3 mRNA increased significantly in the presence of 2 μM and 20 μM DAPT to 927.4%±52.6 (P<0.001, n=6) and 1886.3%±102.4 (P<0.001, n=6) of carrier control, respectively (FIG. 2B).

PTF1A plays a critical role in the formation and spatial organization of the murine exocrine and endocrine pancreas (28) and marks precursor cells that give rise to all exocrine and most endocrine cells (29). In mouse (30) and zebrafish (31), PTF1A is negatively regulated by Notch signaling. Following culture, the level of PTF1A mRNA increased significantly in the presence of 2 μM and 20 μM DAPT to 331.2%±10.8 (P<0.001, n=6) and 391.6%±28.8 (P<0.001, n=6) of carrier control, respectively (FIG. 2C). No significant difference was evident between 2 μM and 20 μM DAPT.

The % of cells expressing NGN3 protein increased significantly in the presence of 2 μM and 20 μM DAPT to 124%±1.1 (P<0.001, n=3) and 152.0%±2.2 (P<0.001, n=3) of carrier control, respectively. No significant effect was evident in treatment with JLK6, a γ-secretase inhibitor, which does not cleave Notch (36)(FIG. 2D). The % of cells expressing NGN3 protein decreased significantly in the presence of 47 μM JAG-1 peptide (JAG), which binds the active site of the Notch ligand Jagged-1, to 39.0%±6.5 (P<0.001, n=3) of cells treated with an equal concentration of negative control peptide comprised of scrambled JAG-1 amino acids (SJAG) (FIG. 2E). The % of NGN3+ cells also decreased significantly in the presence of 500 ng/ml Notch ligand Delta-like ligand 4 (D114), to 60.1%±11.4 percent of carrier control (P<0.05, n=2, data not shown). This decrease is blocked by DAPT, suggesting D114 is working through the canonical Notch signaling pathway.

### Example 4

### NGN3 is Positively Regulated by STAT3 Activation

Like Notch, the JAK-STAT pathway can exert a pleiotropic effect on cell survival, proliferation and fate (32, 33). Crosstalk between these pathways, through Hes1 activation of STAT3, has been described (34). In order to determine if STAT3 activation plays a role in the regulation of NGN3 protein level, cultures were treated for 4 days with PpYLKTK-mts, a STAT3 inhibitor peptide. The % NGN3+ cells decreased significantly in the presence of 0.025 μM, 0.25 μM and 2.5 μM PpYLKTK-mts to 51.8%±8.0 (P<0.001, n=3), 25.3%±5.4 (P<0.001, n=3) and 23.2%±5.7 (P<0.001, n=3) of carrier control, respectively (FIG. 2F).

### Example 5

### NGN3 is Positively Regulated by Epidermal Growth Factor Receptor (EGFR) Activation

AKT and ERK1/2 play roles in several signaling pathways, including a role downstream of the EGFR. In order to determine if EGFR activation plays a role in regulation of NGN3 protein level, cultures were treated for 4 days with the EGFR kinase inhibitor AG1478. The % NGN3+ cells decreased significantly in the presence of 3 nM and 30 nM AG1478 to 69.1%±7.3 (P<0.001, n=5) and 62.3%±8.2 (P<0.001, n=5) of carrier control, respectively (FIG. 2G). Erlotinib, a second EGFR inhibitor, also significantly decreased the % NGN3+ cells at 0.25 μM to 31.0±4.8 (P<0.001, n=1)(data not shown). No exogenous EGF receptor ligands are added to this culture system. However, addition of EGF (20 ng/ml) or TGFα (40 ng/ml) failed to significantly change the % NGN3+ cells.

### Example 6

### NGN3 is Negatively Regulated by Proteasome Degradation

Ubiquitination and proteasome degradation play a role in the post-translational regulation of neurogenins and their binding partners (35, 36). To determine if NGN3 is subject to proteasome degradation, cultures were treated for 4 days with the proteasome inhibitor MG132. The % NGN3+ cells increased significantly in the presence of 1 μM, 10 μM and 20 μM MG132 to 182.8%±14.9 (P<0.001, n=3), 478.8%±33.6 (P<0.001, n=3) and 533.6%±28.9 (P<0.001, n=3) of carrier control, respectively (FIG. 2H). The % NGN3+ cells also increased in the presence of 2.5 μM clasto-lactacystin beta-lactone, a second proteasome inhibitor, to 233.3%±17.4 (P<0.001, n=1) (data not shown).

### Example 7

### Human NGN3 Protein can be Immunoprecipitated from Human Adult Pancreas Extract

In order to verify the identity of proteins visualized and quantified by immunohistochemistry, anti-NGN3 antibody was used to immunoprecipitate NGN3 from D1 collagenase-digested human adult pancreas tissue Immunoprecipitated human proteins migrating at ˜20 KDa were virtually identical to those detected in mouse E14.5 pancreatic epithelia extract (FIG. 2I). No higher molecular weight bands were detected when mouse IgG light chain-specific secondary antibody was used for detection. NGN3 forms a heterodimer with E47 to regulate proendocrine transcription factors such as NEUROD (37). A protein migrating at the apparent molecular weight of E47 (˜75 KDa) co-precipitated with NGN3 on staining with anti-E12/47, whereas proteins migrating at the apparent molecular weights of E12 (˜90 KDa) and E47 were identified in mouse pancreatic epithelia extract (FIG. 2I).

### Example 8

### Viable NGN3+ Cells can be Isolated Based on Co-Expression of CD133

As described herein, several mechanisms controlling the NGN3+ cell population in cultured adult human pancreas tissue have been established. However, to determine the identity and differentiation capacity of this population, methods to isolate viable cells from cultured tissue were required. Adult pancreas tissue is not amenable to the genetic modification required for direct isolation on the basis of NGN3 protein expression or NGN3 promoter activity. The cell surface glycoprotein CD133 is co-expressed with NGN3 in the human fetal pancreas (38) and is expressed in the adult pancreas (39). The possibility that CD133 could be used as a surrogate marker to enrich for NGN3+ cells from adult pancreas tissue was investigated. In biopsied adult pancreas, CD133 is expressed primarily on the apical surface of CK19+ ductal epithelial cells (data not shown). Following culture, most CD133 immunoreactivity surrounded NGN3-labeled nuclei (FIG. 3A-H). No co-expression of CD133 and insulin was detected, and in five density purification fractions from three independent pancreas tissue preparations, an inverse relationship (R²=0.79, n=5) between % islet purity and initial % CD133+ cells was observed (FIG. 31).

CD133 expression in pancreas tissue cultures was sampled as a function of time. On D2-3 the mean±s.e.m % cells expressing CD133 in 6 independent pancreatic tissue preparations was 13.4%±3.0. After 4 days in culture, the mean±s.e.m % CD133+ cells increased to 35.8%±6.1 (P<0.01, n=6), representing a 267.2%±45.6 increase relative to initial levels (FIG. 3J, black bars). After 4 days in culture, the mean±s.e.m total number of CD133+ cells was 296.3%±29.2 of initial levels (P<0.001, n=6) (FIG. 3J, white bars). BrdU incorporation and cleaved caspase-3 levels were measured to determine if the increase in CD133+ resulted from cell proliferation or selective cell death, respectively. On D7 1.9%±0.5 (53/2742 nuclei) of cells were BrdU+ and no co-immunoreactivity for CD133 and BrdU was detected. Only 0.4% (12/2742) of cells were cleaved caspase-3+ on D7. However, by D14>30% (829/2742) of cells were cleaved caspase-3+.

Three approaches were used to characterize the extent of co-expression between NGN3 and CD133 in cultured pancreas tissue. Immunomagnetically-enriched CD133+ cells were cultured in an environment used to support in vitro 13 cell neogenesis from rat exocrine tissue (11). After 12 hrs, 38.8%±5.9 (n=10 fields) of cells adhered, predominantly as isolated single cells. Over the next 8 days, single cells proliferated to form colonies with a mean±s.e.m cell number of 13.3±7.3 (n=10 fields, 3.7 population doublings) then stopped dividing. At this point, 100% (>1000 nuclei examined) of cells were NGN3+.

FACS analysis of pancreatic tissue before and after CD133 immunomagnetic enriched resulted in 35.5% and 92.1% CD133+ cells, respectively (FIG. 3K-L). Subsequent FACS analysis of the CD133-enriched population resulted in 74.1% NGN3+(FIG. 3M). However, parallel immunostaining of the CD133-enriched population immobilized to glass slides resulted in 98.3% (2961/3010 nuclei) NGN3+ cells.

Centroacinar/terminal ductal progenitors were isolated in the mouse pancreas based on ALDH activity (40). Cells from two individual islet preparations were immunolabeled to detect CD133 and then ALDH activity. Cells were gated based on isotype negative control and ALDH inhibitor (DEAB) such that four populations were collected (P2-5) and compared to the unsorted population (US). P2 (2.8% of gated cells) and P3 (45.7%) represent the CD133+/ALDH^{bright} cells, while P4 (13.1%) the CD133+/ALDH− and P5 (17.0%) the CD133−/ALDH^{bright} cells (FIG. 3N). Quantitative RT-PCR from these populations demonstrate that virtually all of the NGN3 mRNA was derived from the P3 population (FIG. 3O). A subsequent experiment replicated the expression of NGN3 by the CD133+/ALDH^{bright} population (36.6% of cells) and demonstrated that the P2 population was a spectral overlap artifact, as it occurs in the absence of CD133 antibody. The CD133+/ALDH− (P4) population may represent efflux of fluorescent substrate that is seen with prolonged room temperature incubation. These data support CD133-immunomagnet enrichment or ALDH FACS as means to obtain populations enriched for NGN3+ cells from pancreas tissue preparations.

### Example 9

### A Subset of CD133+/NGN3+ Cells Form Spherical Aggregates Containing Cells Expressing CPEP, Glucagon (GLU) PDX1 and Chromogranin a (CgA) in Suspension Culture

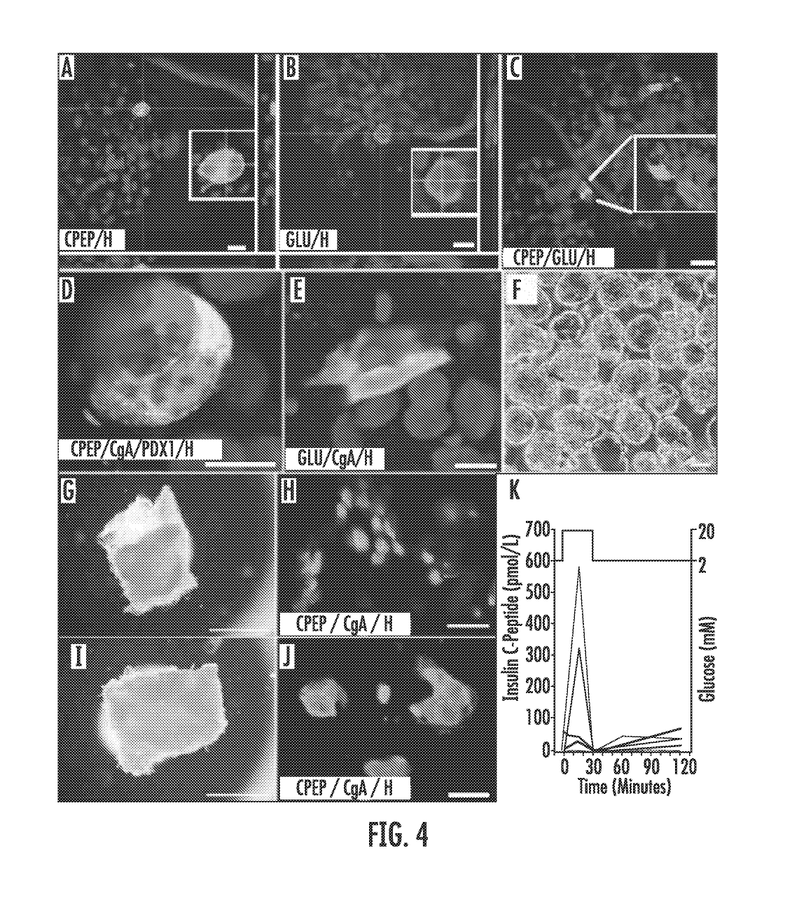

In order to evaluate the capacity of CD133+/NGN3+ cells to proliferate and differentiate in vitro, 5×10⁵ cells enriched to >90% CD133+, were grown in suspension culture. Spherical cell aggregates termed pancospheres formed after 4 days, similar to suspension culture of ALDH^{bright} mouse pancreatic cells (40). After 3 weeks in culture, ˜300 pancospheres with a mean diameter of 162 microns±4.65 were detected (representative image shown in FIG. 4F). The CD133-population failed to form spheres under the same conditions. In three subsequent studies, ˜30% of cells were EdU positive 4 to 5 days after initial plating. Virtually all cells within pancospheres expressed E-cadherin (data not shown). Pancospheres from three independent pancreas tissue cultures were differentiated using an endocrine maturation protocol. Prior to endocrine maturation, NGN3 was but not CPEP+ or GLU+ cells were detected in 15 pancospheres examined. In 3 independent cultures prior to endocrine maturation, a mean±s.e.m of 44.6%±4.9 cells expressed PDX1 localized in the nucleus compared to 2.7%±0.37 cells following endocrine maturation (P<0.001, n=12) (representative image shown in FIG. 4A). Following endocrine maturation, 0.7%±0.2 of cells were CPEP+ in 17 pancospheres carried through endocrine differentiation and maturation. Mean±s.e.m GLU expression was 0.5%±0.2 and mean±s.e.m CgA expression was 0.5%±0. Mycophenolic acid (MPA), an inhibitor of de novo GTP synthesis used clinically to prevent organ transplant rejection and as an inhibitor of angiogenesis, was identified in a recent drug screen to promote precocious endocrine development in zebrafish (43). In a second study of pancospheres from 3 independent cultures, endocrine maturation media was supplemented with 10 μM MPA or DMSO carrier control. In the presence of MPA, 2.9%±0.4 cells were CPEP+ compared to 1.0±0.3 in carrier control pancospheres (P<0.001, n=12). In this study, 1.1%±0.23 cells were GLU+ in the presence of MPA compared to 0.58%±0.2 in carrier control (P>0.05) (representative images shown in FIG. 4 B, C). CPEP+ and GLU+ cells often were present in the same pancosphere, however no co-expression of CPEP and GLU was detected. CgA often was co-expressed with CPEP (FIG. 4D) and GLU (FIG. 4E). Approximately 77% CPEP+/CgA+ cells had detectable levels of PDX1 in the nucleus (representative images of CPEP+/CgA+/PDX1+ cells shown in FIG. 4D). AMY, CK19 and NGN3 expression was not detected in mature pancospheres.

### Example 10

### Single CD133+/NGN3+ Cells are Capable of Clonal Pancosphere Formation

Clonogenic assays were performed to determine if single CD133+/NGN3+ cells form clonal pancospheres. In two experiments, 8.0-11.4% of CD133+/NGN3+ cells underwent clonal expansion and pancosphere formation. Cells from the CD133− fraction formed only loose cell aggregates when plated at 10 cells (2.1%) and 100 cells (13.5%) per well.

### Example 11

### CD133+/NGN3+ Cells Differentiate into CPEP+ Cells in Nanofiber Scaffolds and Release CPEP

In two independent trials, 5×10⁵-1×10⁶ CD133+/NGN3+ cells (>95% CD133+) were suspended in either 100% polyethylene glycol diacrylate (PEG) or 10% Matrigel (MAT) then mixed with electrospun polycaprolactone (PCL) nanofibers. Nanofiber scaffolds were cut into 4 to 6 segments (representative PEG and MAT segments shown in FIGS. 4G and I, respectively) and carried through the endocrine maturation protocol. After 21 days, 74.4%±3.4 (n=6 segments) and 85.6%±4.1 (n=6 segments) cells were CPEP+ in PEG and MAT, respectively. CPEP+ cells in PEG were single or in loosely associated cell clusters (FIG. 4H). CPEP+ cells in MAT scaffolds frequently formed tightly associated multicellular clusters (FIG. 4J). No CgA expression was detected from cells in MAT scaffolds and no GLU or PDX1 expression was detected in cells within PEG or MAT scaffolds. No AMY+ or CK19+ cells were identified in either PEG or MAT scaffolds.

A static CPEP release assay was performed on cells within 6 individual scaffold segments (3 PEG, 3 MAT, ˜2 mm³/segment) 36 days after initial plating. The highest concentration of CPEP produced was 579 pmol CPEP/L and 318 pmol CPEP/L in MAT (FIG. 4K, purple diamond) and PEG (red square), respectively. Peak CPEP release in physiologically high (20 mM) glucose was detected after 15 min. The remaining segments produced peak levels under 100 pmol. Histological analysis of segments used in the static CPEP release assay demonstrated that cells were embedded at a relatively low and highly heterogeneous cell density (FIG. 4H, J are representative of the highest density observed). The 2-scaffold segments producing the highest levels of CPEP in the ELISA had a higher cell density than segments that release low levels of CPEP; one segment (orange circle) had virtually no embedded cells.

### Discussion

NGN3 is Expressed in Human Adult Pancreatic Tissue In Vivo.

NGN3 protein expression was detected in the exocrine tissue of all adult human pancreata examined, but not in adult rodent pancreatic tissue (2). Although the sections of normal pancreas used in this study were non-pathological, they may not accurately reflect NGN3 expression in completely healthy tissue as all patients had some pancreatic lesion that led to surgery. In an effort to minimize the impact of the pancreatic lesion, normal pancreatic parenchyma remote from the lesion were sampled, and pancreata resected for innocuous lesions such as ectopic intrapancreatic spleen were included.

Regulation of NGN3 in Cultured Pancreatic Tissue.

During murine pancreatic development Notch signaling maintains a pancreatic progenitor population by preventing differentiation into endocrine (26, 27) and acinar (31, 44, 45) lineages. The dose-dependent decrease of HES1 and increase of NGN3 mRNA and protein levels following pharmacological inhibition of Notch and decrease in NGN3 protein following treatment with Notch ligands suggest that upstream transcriptional regulation of NGN3 by Notch is active in this tissue. The positive correlation between increased NGN3 protein level and NEUROD1 mRNA levels suggests that endocrine developmental regulation downstream of NGN3 also is intact. Although the percentage of cells expressing NGN3 protein is upregulated in culture, NGN3 mRNA levels were not consistently upregulated and may be due to NGN3 repression of its own promoter (46) as well as effects a post-transcriptional regulation. In mouse, Ngn3 is regulated by microRNA-mediated inhibition (47, 48). Although the human orthologs of the 4 microRNAs known to target Ngn3 were detected, their levels did not change significantly following culture in the 5 samples reported in Table. 1. The short protein half-life of mouse Ngn3 suggests regulation by protein degradation (49, 50). Other members of the neurogenin-family and neurogenin-binding partners are subject to poly-ubiquitination and proteasomal degradation (35, 36). The increased NGN3 protein that is observed following inhibition of proteasome degradation suggests NGN3 in the human pancreas is regulated similarly, perhaps indirectly through Notch signaling.

In addition to Notch, the EGF receptor plays a role in establishing the size of the NGN3+ cell population. This is consistent with the findings that EGF promotes transdifferentiation of mouse acinar cells into insulin secreting cells and that inhibition of EGF receptor signaling blocks the process (10). A role for STAT3 activation in regulation of the NGN3+ cell population has been established, consistent with the finding that inhibition of the JAK2/STAT3 signaling inhibits NGN3 expression and generation of insulin-expressing cells from cultured rat exocrine tissue (51). The extent to which these signaling molecules interconnect to control the NGN3+ cell population, their in vivo ligands and participation of surrounding niche cells in this regulation are yet to be elucidated. However, Notch signaling through activation of STAT3, AKT and mTOR activation has been shown to regulate mouse neural stem cell survival and differentiation (52).

Increased Expression of NGN3 is Due to Metaplastic Transformation.

Expression of essential markers involved in endocrine development and β cell function following adenovirus-mediated expression of NGN3 in human duct cultures (12, 53) suggests that NGN3 has a similar downstream regulatory capacity in adult human tissue as it does during rodent pancreas development. In the adult human pancreas, NGN3 may mark a population of cells that participate in islet neogenesis in response to injury and disease or undergo cell death in the absence of regenerative signals. Enzymatic digestion and culture of pancreas tissue may recapitulate injury signals similar to those triggered by duct ligation, where NGN3 re-expression is required for an increase in β-cell mass (7) or 90% partial pancreatectomy, where transient NGN3 re-expression has been observed (54). Low levels of BrdU incorporation and cleaved caspase 3, as well as an increase in the total number of CD133+/NGN3+ cells, suggest that this accumulation is a result of metaplastic conversion or transdifferentiation rather than arising through proliferation or selective cell survival.

A Subset of CD133+/NGN3+ Cells Have Endocrine Progenitor Capacity.

Identification of CD133 as a surrogate marker for expression of NGN3 was critical for isolation of viable cells. The extent to which the CD133+ and NGN3+ populations overlap was investigated using several different approaches. FACS analysis of CD133+/NGN3+ cell populations suggest that ˜70% of cells are NGN3+, however parallel immunohistochemical staining of immobilized CD133+ cells resulted in >90% NGN3+. In adherent culture, human CD133+ cells undergo a limited number of cell divisions and remain virtually 100% NGN3+ as opposed to the transient expression of NGN3 that is seen following culture of mouse pancreatic CD133+ cells (38), rat exocrine tissue (51) and cells derived from human embryonic stem cells (55). Isolation of CD133 cells by a magnetic bead process captures virtually all of the detectable NGN3 mRNA that is present in cultured pancreatic tissue. Technical limitations in any study of this type leaves open the possibility that small CD133−/NGN3+ and CD133+/NGN3− populations could remain in cultured human pancreatic tissue. The latter such population could mark residual CD133+ luminal duct cells or a cell population prior to accumulation of a detectable level of NGN3 protein. Results from the clonal pancosphere assay suggest that the CD133+/NGN3+ population itself is heterogeneous with respect to proliferative and sphere forming capacity.

Several independent methods were employed to demonstrate that hormone-producing cells resulting from endocrine differentiation were not due to residual β-cell contamination. Following two rounds of CD133-immunomagnetic bead enrichment, no insulin mRNA could be detected after 40 cycles of PCR. No cells expressing CPEP protein were detected in 1536 cells from two CD133 enriched populations whereas a single CD133-depleted population was 36.7% CPEP+(137/373 cells). When CD133+/NGN3+ cells were exposed to maturation media alone, pancospheres did not form and no CPEP+ or GLU+ cells were detected indicating a requirement of aggregation for endocrine differentiation. Prior to endocrine maturation, no CPEP+ or GLU+ cells were identified. Finally, although a very small number of residual endocrine cells may be present in the CD133-enriched population, it is very unlikely that contamination was the source of high levels of CPEP+ cells in biomatrix scaffolds.

Given the prevalence of NGN3 in the adult pancreas, it is likely that NGN3+ cells are present in co-purified exocrine tissue that is present in autologous and, to a lesser extent, allogeneic islet transplants and may play a role in the positive correlation between the number of “ductal epithelial” cells transplanted and long-term successful metabolic outcomes in islet transplant recipients (56). CD133+/NGN3+ cells can be routinely isolated in large numbers from exocrine tissue using methods that are compatible with clinical use, enabling allogeneic and possibly autologous sources if this population is intact in patients with type I diabetes. Furthermore, pharmacological expansion of the NGN3+ cell population in vivo may enable therapeutic islet neogenesis in adult tissue. Finally, a CD133+/NGN3+ population is intact in patients with type I diabetes, it could serve as the basis for new potential modes of therapeutic intervention.

### REFERENCES

1. Lee, J. C., Smith, S. B., Watada, H., Lin, J., Scheel, D., Wang, J., Mirmira, R. G., and German, M. S. 2001. Regulation of the pancreatic pro-endocrine gene neurogenin3. Diabetes 50:928-936.
2. Wang, S., Jensen, J. N., Seymour, P. A., Hsu, W., Dor, Y., Sander, M., Magnuson, M. A., Serup, P., and Gu, G. 2009. Sustained Neurog3 expression in hormone-expressing islet cells is required for endocrine maturation and function. Proc Natl Acad Sci USA 106:9715-9720.
3. Dor, Y., Brown, J., Martinez, O. I., and Melton, D. A. 2004. Adult pancreatic beta-cells are formed by self-duplication rather than stem-cell differentiation. Nature 429:41-46.
4. Desai, B. M., Oliver-Krasinski, J., De Leon, D. D., Farzad, C., Hong, N., Leach, S. D., and Stoffers, D. A. 2007. Preexisting pancreatic acinar cells contribute to acinar cell, but not islet beta cell, regeneration. J Clin Invest 117:971-977.
5. Lipsett, M., and Finegood, D. T. 2002. beta-cell neogenesis during prolonged hyperglycemia in rats. Diabetes 51:1834-1841.
6. Bonner-Weir, S., Baxter, L. A., Schuppin, G. T., and Smith, F. E. 1993. A second pathway for regeneration of adult exocrine and endocrine pancreas. A possible recapitulation of embryonic development. Diabetes 42:1715-1720.
7. Xu, X., D'Hoker, J., Stange, G., Bonne, S., De Leu, N., Xiao, X., Van de Casteele, M., Mellitzer, G., Ling, Z., Pipeleers, D., et al. 2008. Beta cells can be generated from endogenous progenitors in injured adult mouse pancreas. Cell 132:197-207.
8. Zulewski, H., Abraham, E. J., Gerlach, M. J., Daniel, P. B., Moritz, W., Muller, B., Vallejo, M., Thomas, M. K., and Habener, J. F. 2001. Multipotential nestin-positive stem cells isolated from adult pancreatic islets differentiate ex vivo into pancreatic endocrine, exocrine, and hepatic phenotypes. Diabetes 50:521-533.
9. Gershengorn, M. C., Hardikar, A. A., Wei, C., Geras-Raaka, E., Marcus-Samuels, B., and Raaka, B. M. 2004. Epithelial-to-mesenchymal transition generates proliferative human islet precursor cells. Science 306:2261-2264.
10. Minami, K., Okuno, M., Miyawaki, K., Okumachi, A., Ishizaki, K., Oyama, K., Kawaguchi, M., Ishizuka, N., Iwanaga, T., and Seino, S. 2005. Lineage tracing and characterization of insulin-secreting cells generated from adult pancreatic acinar cells. Proc Natl Acad Sci USA 102:15116-15121.
11. Baeyens, L., De Breuck, S., Lardon, J., Mfopou, J. K., Rooman, I., and Bouwens, L. 2005. In vitro generation of insulin-producing beta cells from adult exocrine pancreatic cells. Diabetologia 48:49-57.
12. Heremans, Y., Van De Casteele, M., in't Veld, P., Gradwohl, G., Serup, P., Madsen, O., Pipeleers, D., and Heimberg, H. 2002. Recapitulation of embryonic neuroendocrine differentiation in adult human pancreatic duct cells expressing neurogenin 3. J Cell Biol 159:303-312.
13. Bonner-Weir, S., Taneja, M., Weir, G. C., Tatarkiewicz, K., Song, K. H., Sharma, A., and O'Neil, J. J. 2000. In vitro cultivation of human islets from expanded ductal tissue. Proc Natl Acad Sci USA 97:7999-8004.
14. Yatoh, S., Dodge, R., Akashi, T., Omer, A., Sharma, A., Weir, G. C., and Bonner-Weir, S. 2007. Differentiation of Affinity-Purified Human Pancreatic Duct Cells to {beta}-Cells. Diabetes 56:1802-1809.
15. Zhou, Q., Brown, J., Kanarek, A., Rajagopal, J., and Melton, D. A. 2008. In vivo reprogramming of adult pancreatic exocrine cells to beta-cells. Nature 455:627-632.
16. Swales, N., Martens, G. A., Bonne, S., Heremans, Y., Borup, R., Van de Casteele, M., Ling, Z., Pipeleers, D., Ravassard, P., Nielsen, F., et al. 2012. Plasticity of adult human pancreatic duct cells by neurogenin3-mediated reprogramming. PLoS One 7:e37055.
17. Bhanot, U. K., and Moller, P. 2009. Mechanisms of parenchymal injury and signaling pathways in ectatic ducts of chronic pancreatitis: implications for pancreatic carcinogenesis. Lab Invest 89:489-497.
18. Parsa, I., Longnecker, D. S., Scarpelli, D. G., Pour, P., Reddy, J. K., and Lefkowitz, M. 1985. Ductal metaplasia of human exocrine pancreas and its association with carcinoma. Cancer Res 45:1285-1290.
19. Gmyr, V., Kerr-Conte, J., Belaich, S., Vandewalle, B., Leteurtre, E., Vantyghem, M. C., Lecomte-Houcke, M., Proye, C., Lefebvre, J., and Pattou, F. 2000. Adult human cytokeratin 19-positive cells reexpress insulin promoter factor 1 in vitro: further evidence for pluripotent pancreatic stem cells in humans. Diabetes 49:1671-1680.
20. Gu, D., and Sarvetnick, N. 1993. Epithelial cell proliferation and islet neogenesis in IFN-g transgenic mice. Development 118:33-46.
21. Song, S. Y., Gannon, M., Washington, M. K., Scoggins, C. R., Meszoely, I. M., Goldenring, J. R., Marino, C. R., Sandgren, E. P., Coffey, R. J., Jr., Wright, C. V., et al. 1999. Expansion of Pdxl-expressing pancreatic epithelium and islet neogenesis in transgenic mice overexpressing transforming growth factor alpha. Gastroenterology 117:1416-1426.
22. Wang, R. N., Kloppel, G., and Bouwens, L. 1995. Duct- to islet-cell differentiation and islet growth in the pancreas of duct-ligated adult rats. Diabetologia 38:1405-1411.
23. Bouwens, L. 2004. Islet morphogenesis and stem cell markers. Cell Biochem Biophys 40:81-88.
24. Schwitzgebel, V. M., Scheel, D. W., Conners, J. R., Kalamaras, J., Lee, J. E., Anderson, D. J., Sussel, L., Johnson, J. D., and German, M. S. 2000. Expression of neurogenin3 reveals an islet cell precursor population in the pancreas. Development 127:3533-3542.
25. Dovey, H. F., John, V., Anderson, J. P., Chen, L. Z., de Saint Andrieu, P., Fang, L. Y., Freedman, S. B., Folmer, B., Goldbach, E., Holsztynska, E. J., et al. 2001. Functional gamma-secretase inhibitors reduce beta-amyloid peptide levels in brain. J Neurochem 76:173-181.
26. Jensen, J., Pedersen, E. E., Galante, P., Hald, J., Heller, R. S., Ishibashi, M., Kageyama, R., Guillemot, F., Serup, P., and Madsen, O. D. 2000. Control of endodermal endocrine development by Hes-1. Nat Genet 24:36-44.
27. Apelqvist, A., Li, H., Sommer, L., Beatus, P., Anderson, D. J., Honjo, T., Hrabe de Angelis, M., Lendahl, U., and Edlund, H. 1999. Notch signalling controls pancreatic cell differentiation. Nature 400:877-881.
28. Krapp, A., Knofler, M., Ledermann, B., Burki, K., Berney, C., Zoerkler, N., Hagenbuchle, O., and Wellauer, P. K. 1998. The bHLH protein PTF1-p48 is essential for the formation of the exocrine and the correct spatial organization of the endocrine pancreas. Genes Dev 12:3752-3763.
29. Kawaguchi, Y., Cooper, B., Gannon, M., Ray, M., MacDonald, R. J., and Wright, C. V. 2002. The role of the transcriptional regulator Ptfla in converting intestinal to pancreatic progenitors. Nat Genet 32:128-134.
30. Fukuda, A., Kawaguchi, Y., Furuyama, K., Kodama, S., Horiguchi, M., Kuhara, T., Koizumi, M., Boyer, D. F., Fujimoto, K., Doi, R., et al. 2006. Ectopic pancreas formation in Hes1-knockout mice reveals plasticity of endodermal progenitors of the gut, bile duct, and pancreas. J Clin Invest 116:1484-1493.
31. Esni, F., Ghosh, B., Biankin, A. V., Lin, J. W., Albert, M. A., Yu, X., MacDonald, R. J., Civin, C. I., Real, F. X., Pack, M. A., et al. 2004. Notch inhibits Ptfl function and acinar cell differentiation in developing mouse and zebrafish pancreas. Development 131:4213-4224.
32. Benekli, M., Baer, M. R., Baumann, H., and Wetzler, M. 2003. Signal transducer and activator of transcription proteins in leukemias. Blood 101:2940-2954.
33. Bonni, A., Sun, Y., Nadal-Vicens, M., Bhatt, A., Frank, D. A., Rozovsky, I., Stahl, N., Yancopoulos, G. D., and Greenberg, M. E. 1997. Regulation of gliogenesis in the central nervous system by the JAK-STAT signaling pathway. Science 278:477-483.
34. Kamakura, S., Oishi, K., Yoshimatsu, T., Nakafuku, M., Masuyama, N., and Gotoh, Y. 2004. Hes binding to STAT3 mediates crosstalk between Notch and JAK-STAT signalling. Nat Cell Biol 6:547-554.
35. Vosper, J. M., Fiore-Heriche, C. S., Horan, I., Wilson, K., Wise, H., and Philpott, A. 2007. Regulation of neurogenin stability by ubiquitin-mediated proteolysis. Biochem J 407:277-284.
36. Nie, L., Xu, M., Vladimirova, A., and Sun, X. H. 2003. Notch-induced E2A ubiquitination and degradation are controlled by MAP kinase activities. Embo J 22:5780-5792.
37. Huang, H. P., Liu, M., El-Hodiri, H. M., Chu, K., Jamrich, M., and Tsai, M. J. 2000. Regulation of the pancreatic islet-specific gene BETA2 (neuroD) by neurogenin 3. Mol Cell Biol 20:3292-3307.
38. Sugiyama, T., Rodriguez, R. T., McLean, G. W., and Kim, S. K. 2007. Conserved markers of fetal pancreatic epithelium permit prospective isolation of islet progenitor cells by FACS. Proc Natl Acad Sci USA 104:175-180.
39. Koblas, T., Pektorova, L., Zacharovova, K., Berkova, Z., Girman, P., Dovolilova, E., Karasova, L., and Saudek, F. 2008. Differentiation of CD133-positive pancreatic cells into insulin-producing islet-like cell clusters. Transplant Proc 40:415-418.
40. Rovira, M., Scott, S. G., Liss, A. S., Jensen, J., Thayer, S. P., and Leach, S. D. 2010. Isolation and characterization of centroacinar/terminal ductal progenitor cells in adult mouse pancreas. Proc Natl Acad Sci USA 107:75-80.
41. Hui, H., Khoury, N., Zhao, X., Balkir, L., D'Amico, E., Bullotta, A., Nguyen, E. D., Gambotto, A., and Perfetti, R. 2005. Adenovirus-mediated XIAP gene transfer reverses the negative effects of immunosuppressive drugs on insulin secretion and cell viability of isolated human islets. Diabetes 54:424-433.
42. Johnson, J. D., Ao, Z., Ao, P., Li, H., Dai, L. J., He, Z., Tee, M., Potter, K. J., Klimek, A. M., Meloche, R. M., et al. 2009. Different effects of FK506, rapamycin, and mycophenolate mofetil on glucose-stimulated insulin release and apoptosis in human islets. Cell Transplant 18:833-845.
43. Rovira, M., Huang, W., Yusuff, S., Shim, J. S., Ferrante, A. A., Liu, J. O., and Parsons, M. J. 2011. Chemical screen identifies FDA-approved drugs and target pathways that induce precocious pancreatic endocrine differentiation. Proc Natl Acad Sci USA 108:19264-19269.
44. Hald, J., Hjorth, J. P., German, M. S., Madsen, O. D., Serup, P., and Jensen, J. 2003. Activated Notchl prevents differentiation of pancreatic acinar cells and attenuate endocrine development. Dev Biol 260:426-437.
45. Murtaugh, L. C., Stanger, B. Z., Kwan, K. M., and Melton, D. A. 2003. Notch signaling controls multiple steps of pancreatic differentiation. Proc Natl Acad Sci USA 100:14920-14925.
46. Smith, S. B., Watada, H., and German, M. S. 2004. Neurogenin3 activates the islet differentiation program while repressing its own expression. Mol Endocrinol 18:142-149.
47. Villasenor, A., Chong, D. C., and Cleaver, O. 2008. Biphasic Ngn3 expression in the developing pancreas. Dev Dyn 237:3270-3279.
48. Joglekar, M. V., Parekh, V. S., Mehta, S., Bhonde, R. R., and Hardikar, A. A. 2007. MicroRNA profiling of developing and regenerating pancreas reveal post-transcriptional regulation of neurogenin3. Dev Biol 311:603-612.
49. Barrow, J., Bernardo, A. S., Hay, C. W., Blaylock, M., Duncan, L., Mackenzie, A., McCreath, K., Kind, A. J., Schnieke, A. E., Colman, A., et al. 2005. Purification and Characterization of a Population of EGFP-Expressing Cells from the Developing Pancreas of a Neurogenin3/EGFP Transgenic Mouse. Organogenesis 2:22-27.
50. Miyatsuka, T., Li, Z., and German, M. S. 2009. Chronology of islet differentiation revealed by temporal cell labeling. Diabetes 58:1863-1868.
51. Baeyens, L., Bonne, S., German, M. S., Ravassard, P., Heimberg, H., and Bouwens, L. 2006. Ngn3 expression during postnatal in vitro beta cell neogenesis induced by the JAK/STAT pathway. Cell Death Differ 13:1892-1899.
52. Androutsellis-Theotokis, A., Leker, R. R., Soldner, F., Hoeppner, D. J., Ravin, R., Poser, S. W., Rueger, M. A., Bae, S. K., Kittappa, R., and McKay, R. D. 2006. Notch signalling regulates stem cell numbers in vitro and in vivo. Nature 442:823-826.
53. Gasa, R., Mrejen, C., Leachman, N., Otten, M., Barnes, M., Wang, J., Chakrabarti, S., Mirmira, R., and German, M. 2004. Proendocrine genes coordinate the pancreatic islet differentiation program in vitro. Proc Natl Acad Sci USA 101:13245-13250.
54. Li, W. C., Rukstalis, J. M., Nishimura, W., Tchipashvili, V., Habener, J. F., Sharma, A., and Bonner-Weir, S. Activation of pancreatic-duct-derived progenitor cells during pancreas regeneration in adult rats. J Cell Sci 123:2792-2802.
55. D'Amour, K. A., Bang, A. G., Eliazer, S., Kelly, O. G., Agulnick, A. D., Smart, N. G., Moorman, M. A., Kroon, E., Carpenter, M. K., and Baetge, E. E. 2006. Production of pancreatic hormone-expressing endocrine cells from human embryonic stem cells. Nat Biotechnol.
56. Street, C. N., Lakey, J. R., Shapiro, A. M., Imes, S., Rajotte, R. V., Ryan, E. A., Lyon, J. G., Kin, T., Avila, J., Tsujimura, T., et al. 2004. Islet graft assessment in the Edmonton Protocol: implications for predicting long-term clinical outcome. Diabetes 53:3107-3114.
57. Ricordi, C., Lacy, P. E., Finke, E. H., Olack, B. J., and Scharp, D. W. 1988. Automated method for isolation of human pancreatic islets. Diabetes 37:413-420.
58. Nickoloff, B. J., Qin, J. Z., Chaturvedi, V., Denning, M. F., Bonish, B., and Miele, L. 2002. Jagged-1 mediated activation of notch signaling induces complete maturation of human keratinocytes through NF-kappaB and PPARgamma Cell Death Differ 9:842-855.
59. Clark, G. O., Yochem, R. L., Axelman, J., Sheets, T. P., Kaczorowski, D. J., and Shamblott, M. J. 2007. Glucose responsive insulin production from human embryonic germ (EG) cell derivatives. Biochem Biophys Res Commun 356:587-593.
60. Shamblott, M., Axelman, J., Littlefield, J., Blumenthal, P., Huggins, G., Cui, Y., Cheng, L., and Gearhart, J. 2001. Human embryonic germ cell derivatives express a broad range of developmentally distinct markers and proliferate extensively in vitro. Proc Natl Acad Sci USA 98:113-118.

1. A method for isolating a population of endocrine progenitor cells comprising the steps of:
a. providing a pancreatic tissue sample;
b. isolating cells positive for CD133+; and
c. culturing the isolated cells in defined media for at least about 4 days.

2. The method of claim 1, wherein the isolation step is carried out using immunomagnetic beads.

3. The method of claim 1, wherein the isolation step is accomplished using fluorescence-activate cell sorting (FACS).

4. The method of claim 3, further comprising selecting for Aldefluor-positive cells prior to the culturing step.

5. The method of claim 1, further comprising selecting for SSEA-4+ cells prior to the culturing step.

6. A substantially pure population of CD133+ cells isolated by the method of claim 1, wherein the cells are also NGN3+.

7. A population of cells comprising at least about 90% endocrine progenitor cells, wherein the progenitor cells have the phenotype CD133+.

8. The population of cells of claim 7, wherein the progenitor cells have the phenotype NGN3+.

9. The population of cells of claim 7, wherein the progenitor cells are ALDH+.

10. The population of cells of claim 7, wherein the progenitor cells are SSEA-4+.

11. The population of cells of claim 7, wherein the progenitor cells are capable of clonal pancosphere formation.

12. A method for differentiating human endocrine progenitors comprising the steps of:
a. suspending CD133+/NGN3+ cells in a matrix;
b. mixing the cells-matrix with in a fiber mesh; and
c. differentiating the cells into CPEP+ cells.

13. The method of claim 12, wherein the cells are ALDH+ and/or SSEA-4+.

14. The method of claim 12, wherein the matrix comprises extra cellular matrix extract from human adult islet cells or whole pancreas.

15. The method of claim 12, wherein the matrix comprises a synthetic hydrogel.

16. The method of claim 15, wherein the synthetic hydrogel comprises polyethylene glycol diacrylate.

17. The method of claim 12, wherein the matrix comprises Matrigel.

18. The method of claim 12, wherein the fiber mesh comprises fibers of micro scale.

19. The method of claim 12, wherein the fiber mesh comprises fibers of nano scale.

20. The method of claim 12, wherein the fiber mesh is biodegradable.

21. The method of claim 12, wherein the fiber mesh comprises electrospun polycaprolactone nanofibers.

22. A substantially pure population of human endocrine progenitor cells having the following phenotype: CD133+, NGN3+, and ALDH+.

23. The substantially pure population of human endocrine progenitor cells of claim 22, wherein the cells are also SSEA-4+.

24. The population of cells of claim 22, further exhibiting increased PTF1A expression and increased NEUROD1 expression.

25. A method of treating diabetes in a subject comprising transplanting into the subject a population of endocrine progenitor cells made by the methods of claim 1.

26. A method for treating diabetes in a subject comprising the steps of:
a. culturing a population of cells made by the methods of claim 1 under conditions that differentiate the progenitors into beta cells; and
b. transplanting the beta cells into the subject.
